(19) United States
(12) Patent Application Publication
(10) Pub. No.: US 2008/0318962 A1
(43) Pub. Date: Dec. 25, 2008

(54) Fungicides Based on Nitrogen-Containing Heterocycles

(75) Inventors: Patrick Jelf Crowley, Berkshire (GB); Urs Muller, Basel (CH); Markus Dobler, Cambridge, MA (US); John Williams, Berkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 11/570,980

(22) PCT Filed: Jun. 21, 2005
(86) PCT No.: PCT/EP2005/006687

§ 371 Date: Aug. 20, 2008

(30) Foreign Application Priority Data
Jun. 22, 2004 (GB) 0413955.6

(51) Int. Cl. A01N 43/90; C07D 471/04; A01P 3/00

(52) U.S. Cl. 514/249; 544/350

(57) ABSTRACT

The compound of the general formula (I), wherein W, X, Y, Z, R, R¹ and R² are defined as set forth in the specification, useful as fungicide.

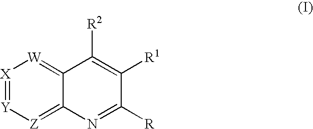

This invention relates to novel pyridine derivatives having a condensed, nitrogen-containing heterocyclic ring, to processes for preparing them, to certain intermediate chemicals used in their manufacture, to compositions containing them and to methods of using them to combat fungi, especially fungal infections of plants.

Derivatives of the nitrogen-containing 5,6 ring systems-1,2,4-triazolo[1,5-a]pyrimidine are known from the patent literature as being useful for controlling phytopathogenic fungi. Examples of recent patent publications include EP-A-1249452, WO 02/051845, WO 02/083676, WO 02/083677, WO 02/088125, WO 02/088126, WO 02/088127. Derivatives of pyridopyrazines are known in the chemical literature, for example from J. Med. Chem. (1968), 11(6), 1216-18, J. Med. Chem. (1970), 13(5), 853-7 and U.S. Pat. No. 3,984,412, but not for agrochemical purposes.

The present invention is concerned with the provision of novel pyridine derivatives having a condensed, nitrogen-containing heterocyclic ring for combating phytopathogenic diseases on plants and harvested food crops.

Thus, according to the present invention, there is provided a compound of the general formula (1):

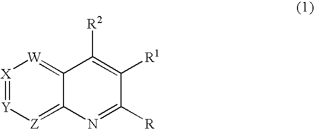

wherein
W, X, Y and Z can be N or CR⁸, with at least one and no more than three of W, X, Y and Z being N, but excluding compounds where W, X, Y═N and Z=CR⁸, and X, Y, Z=N and Z=CR⁸;
R⁸ is H, halo, C₁₋₄ alkyl, C₁₋₄ alkoxy or halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₄₋₅alkynyl, C₂₋₆cycloalkyl, or NR³R⁴
R is H, C₁₋₄ alkyl, halo(C₁₋₄)alkyl, cyano, halogen or NR³R⁴;
R² is halo or NR³R⁴;
R¹ is an aryl or heteroaryl ring R²⁰, of the general formula

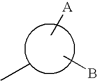

where A can be one to four optional substituents independently selected from halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, C₃₋₆ cycloalkyl(C₁₋₄)alkyl,
and B is at least one or more substituents independently selected from aryl, heteroaryl, aryloxy (except that phenoxy must be substituted), heteroaryloxy, aryl(C₁₋₄)alkoxy (except that benzyloxy must be substituted), heteroaryl(C₁₋₄)alkoxy, arylthio, arylsulphinyl, arylsulphonyl, heteroarylthio, heteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂%)alkynyl, aryl(C₁₋₄)alkyl, heteroaryl(C₁₋₄)alkyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)-alkylthio, C₁₋₄ alkoxy(C₁₋₆)alkyl, C₃₋₆ cycloalkyl, cyano or nitro;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl, heteroaryl(C₁₋₈)alkyl, NR⁵R⁶, usually under the provision that not both R³ and R⁴ are H or NR⁵R⁶, or
R³ and R⁴ together form a C₃₋₇ alkylene or C₃₋₇ alkenylene chain optionally substituted with one or more C₁₋₄ alkyl or C₁₋₄ alkoxy groups, or,
together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl (especially N-methyl) ring; and
R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl or heteroaryl(C₁₋₈)alkyl;
any of the foregoing alkyl, alkenyl, alkynyl or cycloalkyl groups or moieties (other than for R⁸) being optionally substituted with halogen, cyano, C₁₋₆ alkoxy, C₁₋₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino,
any of the foregoing morpholine, thiomorpholine, piperidine, piperazine and pyrrolidine rings being optionally substituted with C₁₋₄ alkyl (especially methyl), and
any of the foregoing aryl or heteroaryl groups or moieties in R³, R⁴, R⁵, R⁶ or R⁸ being optionally substituted with one or more substituents selected from halo, hydroxy, mercapto, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, hydroxy(C₁₋₆)alkyl, C₁₋₄ alkoxy(C₁₋₆)alkyl, C₃₋₆ cycloalkyl, C₃₋₆ cycloalkyl(C₁₋₄)alkyl, phenoxy, benzyloxy, benzoyloxy, cyano, isocyano, thiocyanato, isothiocyanato, nitro, —NR¹³R¹⁴, —NHCOR¹³, —NHCON R¹³R¹⁴, —CONR¹³R¹⁴, —SO₂R¹³, —OSO₂R³, —COR¹³, —CR¹³═NR¹⁴ or —N═CR¹³R¹⁴, in which R¹³ and R¹⁴ are independently hydrogen, C₁₋₄alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy.

The invention includes compounds of the general formula (1) as defined immediately above, preferably compounds in which: C₇ alkylene and C₃₋₇ alkenylene are excluded as chains formed by R³ and R⁴; preferably the C₃₋₆ chain that R³ and R⁴ may form may only be optionally substituted with one or more methyl groups; preferably thiomorpholine, thiomorpholine S-oxide, thiomorpholine S-dioxide and piperazine are excluded as rings that R³ and R⁴ may form; preferably tri(C₁₋₄)alkylsilyl is excluded as a substituent of any alkyl, alkenyl, alkynyl or cycloalkyl group or moiety and any morpholine, piperidine or pyrrolidine ring is unsubstituted.

The compounds of the invention may contain one or more asymmetric carbon atoms and may exist as enantiomers (or as pairs of diastereoisomers) or as mixtures of such. They may also exist as diastereoisomers by virtue of restricted rotation about a bond. However, mixtures of enantiomers or diastereoisomers may be separated into individual isomers or isomer pairs, and this invention embraces such isomers and mixtures thereof in all proportions. It is to be expected that for any given compound, one isomer may be more fungicidally active than another.

Except where otherwise stated, alkyl groups and alkyl moieties of alkoxy, alkylthio, etc., contain from 1 to 8, suitably from 1 to 6 and typically from 1 to 4, carbon atoms in the form of straight or branched chains. Examples are methyl, ethyl, n- and iso-propyl, n-, sec-, iso- and tert-butyl, n-pentyl and n-hexyl. Cycloalkyl groups contain from 3 to 8, typically from 3 to 6, carbon atoms and include bicycloalkyl groups such as the bicyclo[2.2.1]heptyl group. Haloalkyl groups or moieties are typically trichloromethyl or trifluoromethyl or contain a trichloromethyl or trifluoromethyl terminal group. The term fluoroalkyl is an alkyl group substituted by one or more fluorine atoms, as for example trifluoromethyl, difluoroethyl or an alkyl comprising a trifluoromethyl terminal group.

Except where otherwise stated, alkenyl and alkynyl moieties also contain from 2 to 8, suitably from 2 to 6 and typically from 2 to 4, carbon atoms in the form of straight or branched chains. Examples are allyl, 2-methylallyl and propargyl. Optional substituents include halo, typically fluoro. An example of halo-substituted alkenyl is 3,4,4-trifluoro-n-butenyl.

Halo includes fluoro, chloro, bromo and iodo. Most commonly it is fluoro, chloro or bromo and usually fluoro or chloro.

Aryl is usually phenyl but also includes naphthyl, anthryl and phenanthryl.

Heteroaryl is typically a 5- or 6-membered aromatic ring containing one or more O, N or S heteroatoms, which may be fused to one or more other aromatic or heteroaromatic rings, such as a benzene ring. Examples are thienyl, furyl, pyrrolyl, isoxazolyl, oxazolyl, oxadiazolyl, pyrazolyl, imidazolyl, triazolyl, isothiazolyl, tetrazolyl, thiadiazolyl, pyridyl, pyrimidinyl, pyrazinyl, pyridazinyl, triazinyl, benzofuryl, benzothienyl, dibenzofuryl, benzothiazolyl, benzoxazolyl, benzimidazolyl, indolyl, quinolinyl and quinoxalinyl groups and, where appropriate, N-oxides thereof.

The 6,6-ring systems embraced by the general formula (1) are 1,8-naphthyridines (where W, X and Y are all CR⁸ and Z is N), 1,7-naphthyridines (where W, X and Z are all CR⁸ and Y is N), 1,6-naphthyridines (where W, Y and Z are all CR⁸ and X is N), 1,5-naphthyridines (where X, Y and Z are all CR⁸ and W is N), pyrido[2,3-c]pyridazines (where W and X are both CR⁸ and Y and Z are both N), pyrido[2,3-d]pyridazines (where W and Z are both CR⁸ and X and Y are both N), pyrido[3,2-c]pyridazines (where Y and Z are both CR⁸ and W and X are both N), pyrido[2,3-b]pyrazines (where X and Y are both CR⁸ and W and Z are both N), pyrido[2,3-d]pyrimidines (where W and Y are both CR⁸ and X and Z are both N), pyrido[3,2-d]pyrimidines (where X and Z are both CR⁸ and W and Y are both N), pyrido[2,3-e][1,2,4]triazines (where Y is CR⁸ and W, X and Z are all N), and pyrido[3,2-e]-[1,2,4]triazines (where X is CR⁸ and W, Y and Z are all N). Of particular interest are pyrido[2,3-b]pyrazines and pyrido[3,2-e][1,2,4]triazines.

R⁸, which may be the same or different for the CR⁸ values of W, X, Y and Z, is H, halo (for example chloro or bromo), C₁₋₄ alkyl (for example methyl), C₁₋₄ alkoxy (for example methoxy) or halo(C₁₋₄)alkyl (for example trifluoromethyl), CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₄₋₆alkynyl, C₂₋₆cycloalkyl, or NR³R⁴. Usually R⁸ will be H.

One of R and R², preferably R², is NR³R⁴. The other is typically halo, especially chloro or fluoro. In the case of pyrido[2,3-b]pyrazine ring systems, the more active compounds are those where R² is NR³R⁴ and R is chloro or fluoro. R³ is typically C₁₋₈ alkyl (for example ethyl, n-propyl, n-butyl, sec-butyl (the S- or R-isomer or the racemate), isobutyl and tert-butyl), halo(C₁₋₈)alkyl (for example 2,2,2-trifluoroethyl, 2,2,2-trifluoro-1-methylethyl (the S- or R-isomer or the racemate), 2,2,2-trifluoro-1-methylpropyl (the S- or R-isomer or the racemate), 3,3,3-trifluoropropyl and 4,4,4-trifluorobutyl), C₁₋₄ alkoxy(C₁₋₈)alkyl (for example methoxymethyl and methoxy-iso-butyl), C₁₋₄ alkoxyhalo(C₁₋₈)alkyl (for example 2-methoxy-2-trifluoromethylethyl), C₁₋₄ alkylcarbonyl(C₁₋₈)alkyl (for example 1-acetylethyl and 1-tert-butylcarbonylmethyl), C₁₋₄ alkylcarbonylhalo(C₁₋₈)alkyl (for example 1-acetyl-2,2,2-trifluoroethyl), phenyl₍₁₋₄₎alkyl (for example benzyl), C₂₋₈ alkenyl (for example allyl and methylallyl), halo(C₂₋₈)alkenyl (for example 3-methyl-4,4-difluorobut-3-enyl), C₂₋₈ alkynyl (for example propargyl), C₃₋₈ cycloalkyl (for example cyclopropyl, cyclobutyl, cyclopentyl and cyclohexyl) optionally substituted with chloro, fluoro or methyl, C₃₋₈ cyclo-alkyl(C₁₋₄)alkyl (for example cyclopropylmethyl, cyclobutylmethyl, cyclopentylmethyl and cyclohexylmethyl), phenylamino, piperidino or morpholino, the phenyl ring of phenylalkyl or phenylamino being optionally substituted with one, two or three substituents selected from halo (typically fluoro, chloro or bromo), C₁₋₄ alkyl (typically methyl), halo(C₁₋₄)alkyl (typically trifluoromethyl), C₁₋₄ alkoxy (typically methoxy) and halo(C₁₋₄)alkoxy (typically trifluoromethoxy). R⁴ is typically H, C₁₋₄ alkyl (for example ethyl and n-propyl), halo(C₁₋₄)-alkyl (for example 2,2,2-trifluoroethyl) or amino. Alternatively R³ and R⁴ together form a C₄₋₆alkylene chain optionally substituted with methyl, for example 3-methylpentylene, or, together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl (especially N-methyl) ring, in which the morpholine or piperazine rings are optionally substituted with methyl.

Typically R¹ is an aromatic carbocyclic or heterocyclic ring of formula R²⁰, preferably an optionally substituted phenyl, pyridyl or thiazole group, and A is from one to four independent halogen atoms, particularly fluorine and chlorine atoms and especially fluorine atoms, or is from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy), and B is at least one or more of the substituents selected from the group comprising aryl, heteroaryl, aryloxy (except that phenoxy must be substituted), heteroaryloxy, aryl(C₁₋₄)alkoxy (except that benzyloxy must be substituted), heteroaryl(C₁₋₄)alkoxy, arylthio, arylsulphinyl, arylsulphonyl, heteroarylthio, heteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl. Examples are 2,6-difluoro-4-phenyl-phenyl, 2-fluoro-4-phenyl-6-chlorophenyl, 2,5,6-trifluoro-4-phenyl phenyl, 2,4,6-trifluoro-4-phenyl-phenyl, 2-chloro-4-phenyl-phenyl, 2-fluoro-4-phenyl-6-methoxyphenyl, and 2-fluoro-6-trifluoromethyl-4-phenyl-phenyl.

Also of particular interest are compounds where R¹ is an aryl or heteroaryl ring R²⁰ being a pyridyl group and A is from one to three halogen atoms or with from one to three substituents selected from halo (for example fluoro and chloro), C₁₋₄ alkyl (for example methyl), halo(C₁₋₄)alkyl (for example trifluoromethyl), C₁₋₄ alkoxy (for example methoxy) or halo(C₁₋₄)alkoxy (for example trifluoromethoxy) and B is at least one or more substituents selected from aryl, heteroaryl, aryloxy (except that phenoxy must be substituted), heteroaryloxy, aryl(C₁₋₄)alkoxy (except that benzyloxy must be substituted), heteroaryl(C₁₋₄)alkoxy, arylthio, arylsulphinyl, arylsulphonyl, heteroarylthio, heteroarylsulphinyl, heteroarylsulphonyl, aryl(C₂₋₄)alkenyl, aryl (C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl. Examples are 3-fluoro-5-phenylpyrid-2-yl, 3-chloro-5-phenylpyrid-2-yl and 3,5-difluoro-4-phenylpyrid-2-yl.

In one aspect the invention provides a compound of the general formula (1) wherein W and Z are N and the other two are CR⁸, or W, Y and Z are N and X is CR⁸, or W, X and Z are N and Y is CR⁸;

R⁸ is H, halo, C₁₋₄ alkyl, C₁₋₄ alkoxy or halo(C₁₋₄)alkyl; one of R and R² (preferably R²) is NR³R⁴ and the other is halo;
R¹ is an aryl or heteroaryl ring R²⁰, and A is a substituent selected from halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄ alkoxy(C₁₋₆)alkyl, C₃₋₆ cycloalkyl, C₃₋₆ cycloalkyl(C₁₋₄)alkyl, and B is at least one or more substituents selected from aryl, heteroaryl, aryloxy (except that phenoxy must be substituted), heteroaryloxy, aryl(C₁₋₄)alkoxy (except that benzyloxy must be substituted), heteroaryl(C₁₋₄)alkoxy, arylthio, arylsulphinyl, arylsulphonyl, heteroarylthio, beteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)-alkylthio, C₁₋₄ alkoxy(C₁₋₆)alkyl, C₃₋₆ cycloalkyl, cyano or nitro;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl, heteroaryl(C₁₋₈)alkyl, NR⁵R⁶, provided that at least one of R³ and R⁴ is NR⁵R⁶, or
R³ and R⁴ together form a C₃₋₇ alkylene or C₃₋₇ alkenylene chain optionally substituted with one or more C₁₋₄ alkyl or C₁₋₄ alkoxy groups, or,
together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl (especially N-methyl) ring; and
R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl or heteroaryl(C₁₋₈)alkyl;
any of the foregoing alkyl, alkenyl, alkynyl or cycloalkyl groups or moieties (other than for R⁸) being optionally substituted with halogen, cyano, C₁₋₆ alkoxy, C₁₋₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino, any of the foregoing morpholine, thiomorpholine, piperidine, piperazine and pyrrolidine rings being optionally substituted with C₁₋₄ alkyl (especially methyl). Of particular interest are compounds where W and Z are both N and X and Y are both CH.

In a further embodiment, the invention includes a compound of the general formula (1) as defined immediately above except that: C₇ alkylene and C₃₋₇ alkenylene are excluded as chains formed by R³ and R⁴; the C₃₋₆ chain that R³ and R⁴ may form may only be optionally substituted with one or more methyl groups; thiomorpholine, thiomorpholine S-oxide, thiomorpholine S-dioxide and piperazine are excluded as rings that R³ and R⁴ may form; tri(C₁₋₄)alkylsilyl is excluded as a substituent of any alkyl, alkenyl, alkynyl or cycloalkyl group or moiety, and any morpholine, piperidine or pyrrolidine ring is unsubstituted.

In yet a further embodiment of the present invention, the compound of formula 1 the values of the substituents are defined as follows:

R³ is C₁₋₈ alkyl, halo(C₁₋₈)alkyl, haloC₁₋₄ alkoxy(C₁₋₈)alkyl, C₁₋₄ alkoxyhalo(C₁₋₈)alkyl, CIA alkoxycarbonyl(C₁₋₈)alkyl, C₁₋₄ alkoxycarbonylhalo(C₁₋₈)alkyl, phenyl(₁₋₄)alkyl, C₂₋₈ alkenyl, halo(C₂₋₈)alkenyl, C₂₋₈ alkynyl, C₃₋₈ cycloalkyl optionally substituted with chloro, fluoro or methyl, C₃₋₈ cycloalkyl(C₁₋₄)alkyl, phenylamino, piperidino or morpholino, the phenyl ring of phenylalkyl or phenylamino being optionally substituted with one, two or three substituents selected from halo, C₁₋₄ alkyl, halo(C₁₋₄)alkyl, C₁₋₄ alkoxy and halo-(C₁₋₄)alkoxy; and R⁴ is H, C₁₋₄ alkyl, halo(C₁₋₄)alkyl or amino, or R³ and R⁴ together form a C₃₋₇ alkylene or alkenylene chain optionally substituted with methyl, or, together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl (especially N-methyl) ring, in which the morpholine or piperazine rings are optionally substituted with methyl. Of particular interest are compounds in which R is halo, and/or R² is NR³R⁴, where NR³R⁴ are as defined above, and/or W and Z are N and X and Y are CH.

A further embodiment of the invention provides a compound of formula (1) wherein W, X and Z are N and Y is CR⁸;

R⁸ is H or halo; R is halo and R² is NR³R⁴; R¹ is an aryl or heteroaryl ring R²⁰ and A is a substituent selected from halo, C₁₋₆ alkyl, C₂₋₆ alkenyl, C₂₋₆ alkynyl, and B is one more substituent selected from aryl, heteroaryl, aryloxy (except that phenoxy must be substituted), heteroaryloxy, arylthio, arylsulphinyl, arylsulphonyl, heteroarylthio, heteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, cyano or nitro; R³ and R⁴ are independently H, C₁₋₈ alkyl, C₁₋₈ fluoroalkyl, C₁₋₈ perfluoroalkyl C₂₋₈ alkenyl, C₂₋₈ alkynyl, aryl, C₃₋₈ cycloalkyl, heteroaryl. Of particular interest are compounds of the general formula (1) as defined immediately above except that: C₇ alkylene and C₃₋₇ alkenylene are excluded as chains formed by R³ and R⁴; tri(C₁₋₄)alkylsilyl is excluded as a substituent of any alkyl, alkenyl, alkynyl or cycloalkyl group or moiety, and any morpholine, piperidine or pyrrolidine ring is unsubstituted.
In a further embodiment of the invention as defined immediately above, the compounds of formula (1) are compounds in which W, X and Z are N, and the respective other ring members are CH;
R is chloro or fluoro;

### R² is NR³R⁴;

R¹ is an aryl or heteroaryl ring of formula R²⁰;
R²⁰ is 4-substituted 2,6-difluorophenyl, 4-substituted 2,3,6-trifluorophenyl, 3-substituted 2,4,6-trifluorophenyl, 4-substituted 2-chloro-6-fluorophenyl, 4-substituted 2-chlorophenyl, 5-substituted 3-fluoropyrid-2-yl, 5-substituted 3-chloropyrid-2-yl, 2-substituted 4-chloro-thiazol-5-yl, 2-substituted 4-fluoro-thiazol-5-yl;
R³ is hydrogen, methyl, ethyl, 1,1,1-trifluoroethyl, 2-methylpropen-3-yl;
R⁴ is prop-2-yl, but-2-yl, 2-methylprop-3-yl, 2-methylbut-3-yl, 1,1,1-trifluoroprop-2-yl, 1,1,1-trifluoroethyl, 1,1,1-trifluorobut-2-yl, 1,1,1-trifluoro-3-methyl-but-2-yl, 2-methylpent-4-yl, 1,1,1-trifluoro-4-methylpent-2-yl, 1,1,1-trifluoro-3-methylpent-2-yl, 3-methylpent-2-yl, 1,1,-difluorocyclopent-2-yl, heptafluoroprop-1-yl, nonafluorobut-1-yl, 1-carboxyethyl-2.methylprop-1-yl, 1-carboxymethyl-2-methylprop-1-yl, 1,1,1,-trifluoro-2-carboxyethyl-2-ethyl, 1,1,-trifluoro-2-carboxymethyl 2-ethyl;
B is phenyl, 4-fluorophenyl, 2-fluorophenyl, 3-fluorophenyl, 4-chlorophenyl, 2-chlorophenyl, 3-chlorophenyl, 2-pyridyl, 3-pyridyl, 4-pyridyl, 5-fluoro-2-pyridyl, 6-fluoro-3-pyridyl, 2-fluoro-4-pyridyl, 2-phenylethenyl, 2-(4-fluorophenyl)ethenyl, phenylethynyl, (4-methylphenyl)ethynyl, (4-fluorophenyl)ethynyl, 4-fluorophenoxy, 2-fluorophenoxy, 3-fluorophenoxy, phenylthio, phenylsulphinyl, phenylsulphonyl; or, in another embodiment of the invention, W, X and Z are N, and the respective other ring members are CH;
R is chloro or fluoro;

### R² is NR³R⁴;

R¹ is an aryl or heteroaryl ring of formula R²⁰;
R²⁰ is 4-substituted 2,6-difluorophenyl, 4-substituted 2-chloro-6-fluorophenyl, 4-substituted 2-chlorophenyl, 5-substituted 3-fluoropyrid-2-yl, 5-substituted 3-chloropyrid-2-yl;
R³ is hydrogen, ethyl, 2-methylpropen-3-yl;
R⁴ is prop-2-yl, but-2-yl, 2-methylprop-3-yl, 2-methylbut-3-yl, 1,1,1-trifluoroprop-2-yl, 1,1,1-trifluoroethyl, 1,1,1-trifluorobut-2-yl, 1,1,1-trifluoro-3-methyl-but-2-yl; 1,1,1-trifluoro-4-methylpent-2-yl, 1,1,1-trifluoro-3-methylpent-2-yl, 1,1,-difluorocyclopent-2-yl, heptafluoroprop-1-yl, 1-carboxyethyl-2-methylprop-1-yl, 1,1,1,-trifluoro-2-carboxyethyl-2-ethyl;

B is phenyl, 4-fluorophenyl, 4-chlorophenyl, 5-fluoro-2-pyridyl, 6-fluoro-3-pyridyl, 2-phenylethenyl, 2-(4-fluorophenyl)ethenyl, (4-methylphenyl)ethynyl, (4-fluorophenyl)ethynyl, 4-fluorophenoxy, phenylthio, phenylsulphinyl (or benzensulphinyl), phenylsulphonyl (or benzenesulphonyl).

Yet another aspect of the invention provides a compound of formula (1) wherein W and Z are N and the other two are CR⁸, or W, Y and Z are N and X is CR⁸,

R⁸ is H or halo; R is halo and R² is NR³R⁴; R¹ is an aryl or heteroaryl ring R²⁰ and A is a substituent selected from halo, C₁₋₆ alkyl, C₂₋₆ alkenyl, C₂₋₆ alkynyl, and B is one more substituent selected from aryl, heteroaryl, aryloxy (except that phenoxy must be substituted), heteroaryloxy, arylthio, arylsulphinyl, arylsulphonyl, heteroarylthio, heteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl, with any of the forgoing aryl or heteroaryl substituents being optionally substituted with halo, C₁₋₆ alkyl, C₂₋₆ alkenyl, C₂₋₆ alkynyl, C₁₋₆ alkoxy, cyano or nitro; R³ and R⁴ are independently H, C₁₋₈ alkyl, C₁₋₈ fluoroalkyl, C₁₋₈ perfluoroalkyl, C₂₋₈ alkenyl, C₂₋₈ alkynyl, aryl, C₃₋₈ cycloalkyl, heteroaryl.

In a further embodiment, the invention includes a compound of the general formula (1) as defined immediately above except that: C₇ alkylene and C₃₋₇ alkenylene are excluded as chains formed by R³ and R⁴; tri(C₁₋₄)alkylsilyl is excluded as a substituent of any alkyl, alkenyl, alkynyl or cycloalkyl group or moiety, and any morpholine, piperidine or pyrrolidine ring is unsubstituted.

In a further embodiment of the invention, in the compounds of formula (1) are compounds in which W and Z are N, or W, Y and Z are N, and the respective other ring members are CH;

R is chloro or fluoro;

### R² is NR³R⁴;

R¹ is an aryl or heteroaryl ring of formula R²⁰;
R²⁰ is 4-substituted-2,6-difluorophenyl, 4-substituted 2,3,6-trifluorophenyl, 3-substituted 2,4,6-trifluorophenyl, 4-substituted 2-chloro-6-fluorophenyl, 4-substituted 2-chlorophenyl, 5-substituted 3-fluoropyrid-2-yl, 5-substituted 3-chloropyrid-2-yl, 2-substituted 4-chloro-thiazol-5-yl, 2-substituted 4-fluoro-thiazol-5-yl;
R³ is hydrogen, methyl, ethyl, 1,1,1-trifluoroethyl, 2-methylpropen-3-yl;
R⁴ is prop-2-yl, but-2-yl, 2-methylprop-3-yl, 2-methylbut-3-yl, 1,1,1-trifluoroprop-2-yl, 1,1,1-trifluoroethyl, 1,1,1-trifluorobut-2-yl, 1,1,1-trifluoro-3-methyl-but-2-yl; 2-methylpent-4-yl, 1,1,1-trifluoro-4-methylpent-2-yl, 1,1,1-trifluoro-3-methylpent-2-yl, 3-methylpent-2-yl, 1,1,-difluorocyclopent-2-yl, heptafluoroprop-1-yl, nonafluorobut-1-yl, 1-carboxyethyl-2.methylprop-1-yl, 1-carboxymethyl-2-methylprop-1-yl, 1,1,1,-trifluoro-2-carboxyethyl-2-ethyl, 1,1,1,-trifluoro-2-carboxymethyl 2-ethyl;
B is phenyl, 4-fluorophenyl, 2-fluorophenyl, 3-fluorophenyl, 4-chlorophenyl, 2-chlorophenyl, 3-chlorophenyl, 2-pyridyl, 3-pyridyl, 4-pyridyl, 5-fluoro-2-pyridyl, 6-fluoro-3-pyridyl, 2-fluoro-4-pyridyl, 2-phenylethenyl, 2-(4-fluorophenyl)ethenyl, phenylethynyl, (4-methylphenyl)ethynyl, (4-fluorophenyl)ethynyl, 4-fluorophenoxy, 2-fluorophenoxy, 3-fluorophenoxy, phenylthio, phenylsulphinyl, phenylsulphonyl, or Benzenesulfonyl;
or, in another embodiment of the invention,
W and Z are N, or W, Y and Z are N, and the respective other ring members are CH;
R is chloro or fluoro;

### R² is NR³R⁴;

R¹ is an aryl or heteroaryl ring of formula R²⁰;
R²⁰ is 4-substituted 2,6-difluorophenyl, 4-substituted 2-chloro-6-fluorophenyl, 4-substituted 2-chlorophenyl, 5-substituted 3-fluoropyrid-2-yl, 5-substituted 3-chloropyrid-2-yl;
R³ is hydrogen, ethyl, 2-methylpropen-3-yl;
R⁴ is prop-2-yl, but-2-yl, 2-methylprop-3-yl, 2-methylbut-3-yl, 1,1,1-trifluoroprop-2-yl, 1,1,1-trifluoroethyl, 1,1,1-trifluorobut-2-yl, 1,1,1-trifluoro-3-methyl-but-2-yl; 1,1,1-trifluoro-4-methylpent-2-yl, 1,1,1-trifluoro-3-methylpent-2-yl, 1,1,-difluorocyclopent-2-yl, heptafluoroprop-1-yl, 1-carboxyethyl-2-methylprop-1-yl, 1,1,1,-trifluoro-2-carboxyethyl-2-ethyl;
B is phenyl, 4-fluorophenyl, 4-chlorophenyl, 5-fluoro-2-pyridyl, 6-fluoro-3-pyridyl, 2-phenylethenyl, 2-(4-fluorophenyl)ethenyl, (4-methylphenyl)ethynyl, (4-fluorophenyl)ethynyl, 4-fluorophenoxy, phenylthio, phenylsulphinyl (or benzensulphinyl), phenylsulphonyl (or benzenesulphonyl).

Compounds that form part of the invention are illustrated in Tables 1 to 27 below. Characterising data are given later in the Examples. Single compounds are assigned the number of the table, followed by the number of the combination of substituents as in Table 1. For example, compound 22.005 is the compound as described in Table 22, wherein the substituents defined therein are combined with the substituents as defined in Table 1, position No. 5. In Table 1 to 27 the compounds have the general formula (2) as depicted below.

TABLE 1

| | |
|---|---|
| | (2) |
| 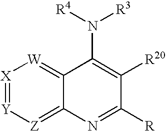 | |

| Position | | | | |
|---|---|---|---|---|
| No. | R | R³ | R⁴ | R²⁰ |
| 001 | Cl | H | —CH(CH₃)₂ | 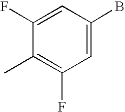 |
| 002 | Cl | H | —CH(CH₃)CH₂CH₃ | 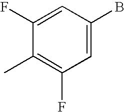 |
| 003 | Cl | H | —CH₂CH(CH₃)₂ | 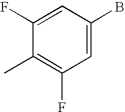 |
| 004 | Cl | H | —CH(CH₃)CH(CH₃)₂ | 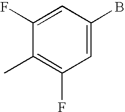 |
| 005 | Cl | H | —CH(CH₃)(CF₃) | 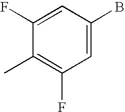 |
| 006 | Cl | H | —CH₂CF₃ | 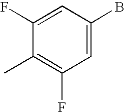 |
| 007 | Cl | H | —CH(CF₃)CH₂CH₃ | 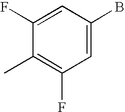 |
| 008 | Cl | H | —CH(CF₃)CH(CH₃)₂ | 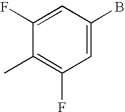 |
| 009 | Cl | H | —CH(CF₃)CH₂CH(CH₃)₂ | 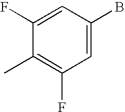 |
| 010 | Cl | H | —CH(CF₃)CH(CH₃)CH₂CH₃ | 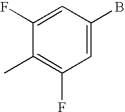 |
| 011 | Cl | H | 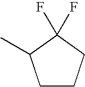 | 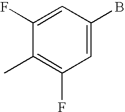 |
| 012 | Cl | H | —CF₂CF₂CF₃ | 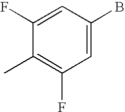 |
| 013 | Cl | H | —CH(CH[CH₃]₂)COOCH₂CH₃ | 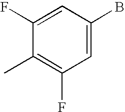 |
| 014 | Cl | H | —CH(CF₃)COOCH₂CH₃ | 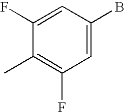 |
| 015 | Cl | H | —CH(CH₃)₂ | 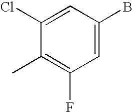 |
| 016 | Cl | H | —CH(CH₃)CH₂CH₃ | 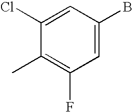 |
| 017 | Cl | H | —CH₂CH(CH₃)₂ | 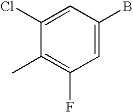 |
| 018 | Cl | H | —CH(CH₃)CH(CH₃)₂ | 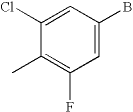 |
| 019 | Cl | H | —CH(CH₃)(CF₃) | 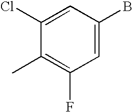 |
| 020 | Cl | H | —CH₂CF₃ | 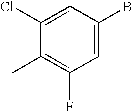 |
| 021 | Cl | H | —CH(CF₃)CH₂CH₃ | 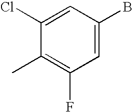 |
| 022 | Cl | H | —CH(CF₃)CH(CH₃)₂ | 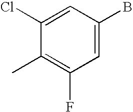 |
| 023 | Cl | H | —CH(CF₃)CH₂CH(CH₃)₂ | 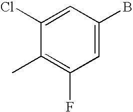 |
| 024 | Cl | H | —CHH(CF₃)CH(CH₃)CH₂CH₃ | 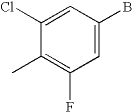 |
| 025 | Cl | H | 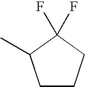 | 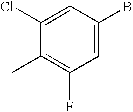 |
| 026 | Cl | H | —CF₂CF₂CF₃ | 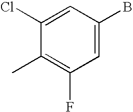 |
| 027 | Cl | H | —CH(CH[CH₃]₂)COOCH₂CH₃ | 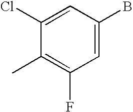 |
| 028 | Cl | H | —CH(CF₃)COOCH₂CH₃ | 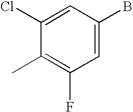 |
| 029 | Cl | H | —CH(CH₃)₂ | 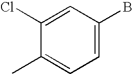 |
| 030 | Cl | H | —CH(CH₃)CH₂CH₃ | 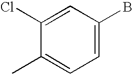 |
| 031 | Cl | H | —CH₂CH(CH₃)₂ | 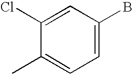 |
| 032 | Cl | H | —CH(CH₃)CH(CH₃)₂ | 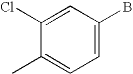 |
| 033 | Cl | H | —CH(CH₃)(CF₃) | 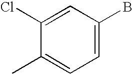 |
| 034 | Cl | H | —CH₂CF₃ | 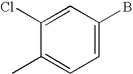 |
| 035 | Cl | H | —CH(CF₃)CH₂CH₃ | 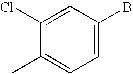 |
| 036 | Cl | H | —CH(CF₃)CH(CH₃)₂ | 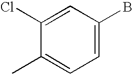 |
| 037 | Cl | H | —CH(CF₃)CH₂CH(CH₃)₂ | 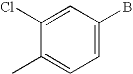 |
| 038 | Cl | H | —CH(CF₃)CH(CH₃)CH₂CH₃ | 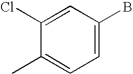 |
| 039 | Cl | H | 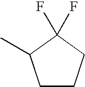 | 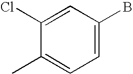 |
| 040 | Cl | H | —CF₂CF₂CF₃ | 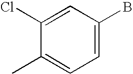 |
| 041 | Cl | H | —CH(CH[CH₃]₂)COOCH₂CH₃ | 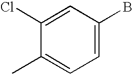 |
| 042 | Cl | H | —CH(CF₃)COOCH₂CH₃ | 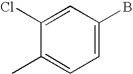 |
| 043 | Cl | H | —CH(CH₃)₂ | 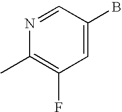 |
| 044 | Cl | H | —CH(CH₃)CH₂CH₃ | 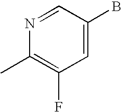 |
| 045 | Cl | H | —CH₂CH(CH₃)₂ | 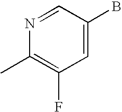 |
| 046 | Cl | H | —CH(CH₃)CH(CH₃)₂ | 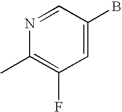 |
| 047 | Cl | H | —CH(CH₃)(CF₃) | 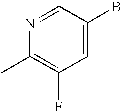 |
| 048 | Cl | H | —CH₂CF₃ | 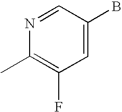 |
| 049 | Cl | H | —CH(CF₃)CH₂CH₃ | 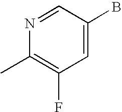 |
| 050 | Cl | H | —CH(CF₃)CH(CH₃)₂ | 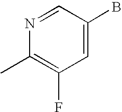 |
| 051 | Cl | H | —CH(CF₃)CH₂CH(CH₃)₂ | 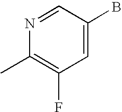 |
| 052 | Cl | H | —CH(CF₃)CH(CH₃)CH₂CH₃ | 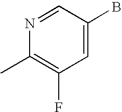 |
| 053 | Cl | H | 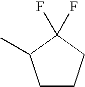 | 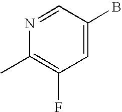 |
| 054 | Cl | H | —CF₂CF₂CF₃ | 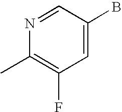 |
| 055 | Cl | H | —CH(CH[CH₃]₂)COOCH₂CH₃ | 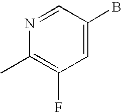 |
| 056 | Cl | H | —CH(CF₃)COOCH₂CH₃ | 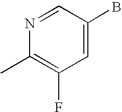 |
| 057 | Cl | H | —CH(CH₃)₂ | 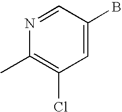 |
| 058 | Cl | H | —CH(CH₃)CH₂CH₃ | 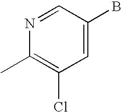 |
| 059 | Cl | H | —CH₂CH(CH₃)₂ | 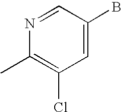 |
| 060 | Cl | H | —CH(CH₃)CH(CH₃)₂ | 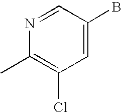 |
| 061 | Cl | H | —CH(CH₃)(CF₃) | 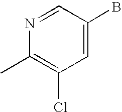 |
| 062 | Cl | H | —CH₂CF₃ | 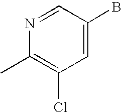 |
| 063 | Cl | H | —CH(CF₃)CH₂CH₃ | 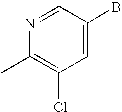 |
| 064 | Cl | H | —CH(CF₃)CH(CH₃)₂ | 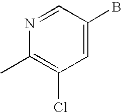 |
| 065 | Cl | H | —CH(CF₃)CH₂CH(CH₃)₂ | 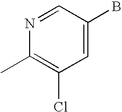 |
| 066 | Cl | H | —CH(CF₃)CH(CH₃)CH₂CH₃ | 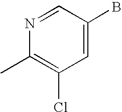 |
| 067 | Cl | H | 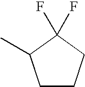 | 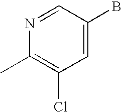 |
| 068 | Cl | H | —CF₂CF₂CF₃ | 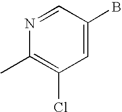 |
| 069 | Cl | H | —CH(CH[CH₃]₂)COOCH₂CH₃ | 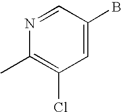 |
| 070 | Cl | H | —CH(CF₃)COOCH₂CH₃ | 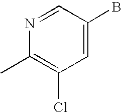 |
| 071 | Cl | CH₂CH₃ | —CH(CH₃)₂ | 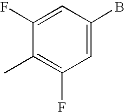 |
| 072 | Cl | CH₂CH₃ | —CH(CH₃)CH₂CH₃ | 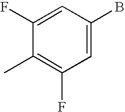 |
| 073 | Cl | CH₂CH₃ | —CH₂CH(CH₃)₂ | 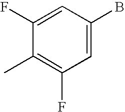 |
| 074 | Cl | CH₂CH₃ | —CH(CH₃)CH(CH₃)₂ | 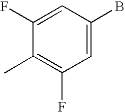 |
| 075 | Cl | CH₂CH₃ | —CH(CH₃)(CF₃) | 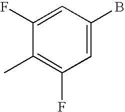 |
| 076 | Cl | CH₂CH₃ | —CH₂CF₃ | 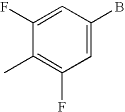 |
| 077 | Cl | CH₂CH₃ | —CH(CF₃)CH₂CH₃ | 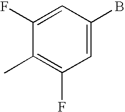 |
| 078 | Cl | CH₂CH₃ | —CH(CF₃)CH(CH₃)₂ | 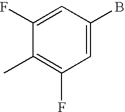 |
| 079 | Cl | CH₂CH₃ | —CH(CF₃)CH₂CH(CH₃)₂ | 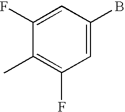 |
| 080 | Cl | CH₂CH₃ | —CH(CF₃)CH(CH₃)CH₂CH₃ | 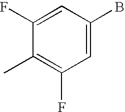 |
| 081 | Cl | CH₂CH₃ | 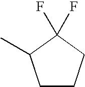 | 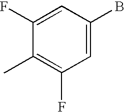 |
| 082 | Cl | CH₂CH₃ | —CF₂CF₂CF₃ | 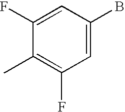 |
| 083 | Cl | CH₂CH₃ | —CH(CH[CH₃]₂)COOCH₂CH₃ | 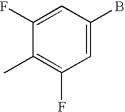 |
| 084 | Cl | CH₂CH₃ | —CH(CF₃)COOCH₂CH₃ | 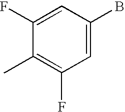 |
| 085 | Cl | CH₂CH₃ | —CH(CH₃)₂ | 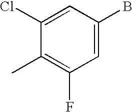 |
| 086 | Cl | CH₂CH₃ | —CH(CH₃)CH₂CH₃ | 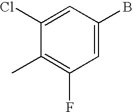 |
| 087 | Cl | CH₂CH₃ | —CH₂CH(CH₃)₂ | 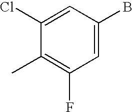 |
| 088 | Cl | CH₂CH₃ | —CH(CH₃)CH(CH₃)₂ | 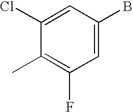 |
| 089 | Cl | CH₂CH₃ | —CH(CH₃)(CF₃) | 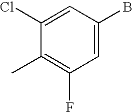 |
| 090 | Cl | CH₂CH₃ | —CH₂CF₃ | 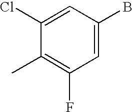 |
| 091 | Cl | CH₂CH₃ | —CH(CF₃)CH₂CH₃ | 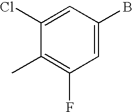 |
| 092 | Cl | CH₂CH₃ | —CH(CF₃)CH(CH₃)₂ | 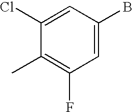 |
| 093 | Cl | CH₂CH₃ | —CH(CF₃)CH₂CH(CH₃)₂ | 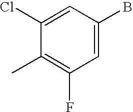 |
| 094 | Cl | CH₂CH₃ | —CH(CF₃)CH(CH₃)CH₂CH₃ | 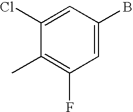 |
| 095 | Cl | CH₂CH₃ | 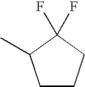 | 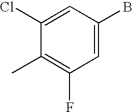 |
| 096 | Cl | CH₂CH₃ | —CF₂CF₂CF₃ | 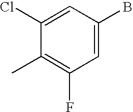 |
| 097 | Cl | CH₂CH₃ | —CH(CH[CH₃]₂)COOCH₂CH₃ | 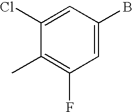 |
| 098 | Cl | CH₂CH₃ | —CH(CF₃)COOCH₂CH₃ | 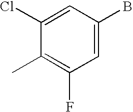 |
| 099 | Cl | CH₂CH₃ | —CH(CH₃)₂ | 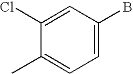 |
| 100 | Cl | CH₂CH₃ | —CH(CH₃)CH₂CH₃ | 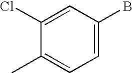 |
| 101 | Cl | CH₂CH₃ | —CH₂CH(CH₃)₂ | 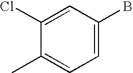 |
| 102 | Cl | CH₂CH₃ | —CH(CH₃)CH(CH₃)₂ | 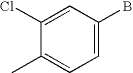 |
| 103 | Cl | CH₂CH₃ | —CH(CH₃)(CF₃) | 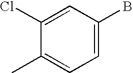 |
| 104 | Cl | CH₂CH₃ | —CH₂CF₃ | 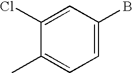 |
| 105 | Cl | CH₂CH₃ | —CH(CF₃)CH₂CH₃ | 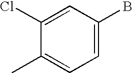 |
| 106 | Cl | CH₂CH₃ | —CH(CF₃)CH(CH₃)₂ | 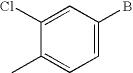 |
| 107 | Cl | CH₂CH₃ | —CH(CF₃)CH₂CH(CH₃)₂ | 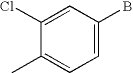 |
| 108 | Cl | CH₂CH₃ | —CH(CF₃)CH(CH₃)CH₂CH₃ | 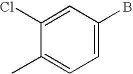 |
| 109 | Cl | CH₂CH₃ | 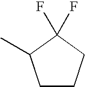 | 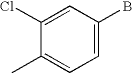 |
| 110 | Cl | CH₂CH₃ | —CF₂CF₂CF₃ | 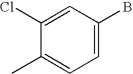 |
| 111 | Cl | CH₂CH₃ | —CH(CH[CH₃]₂)COOCH₂CH₃ | 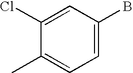 |
| 112 | Cl | CH₂CH₃ | —CH(CF₃)COOCH₂CH₃ | 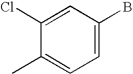 |
| 113 | Cl | CH₂CH₃ | —CH(CH₃)₂ | 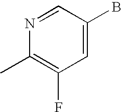 |
| 114 | Cl | CH₂CH₃ | —CH(CH₃)CH₂CH₃ | 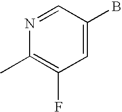 |
| 115 | Cl | CH₂CH₃ | —CH₂CH(CH₃)₂ | 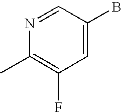 |
| 116 | Cl | CH₂CH₃ | —CH(CH₃)CH(CH₃)₂ | 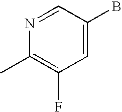 |
| 117 | Cl | CH₂CH₃ | —CH(CH₃)(CF₃) | 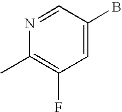 |
| 118 | Cl | CH₂CH₃ | —CH₂CF₃ | 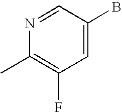 |
| 119 | Cl | CH₂CH₃ | —CH(CF₃)CH₂CH₃ | 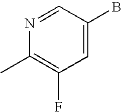 |
| 120 | Cl | CH₂CH₃ | —CH(CF₃)CH(CH₃)₂ | 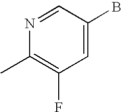 |
| 121 | Cl | CH₂CH₃ | —CH(CF₃)CH₂CH(CH₃)₂ | 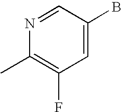 |
| 122 | Cl | CH₂CH₃ | —CH(CF₃)CH(CH₃)CH₂CH₃ | 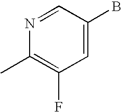 |
| 123 | Cl | CH₂CH₃ | 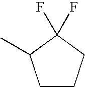 | 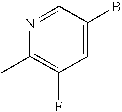 |
| 124 | Cl | CH₂CH₃ | —CF₂CF₂CF₃ | 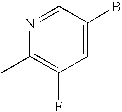 |
| 125 | Cl | CH₂CH₃ | —CH(CH[CH₃]₂)COOCH₂CH₃ | 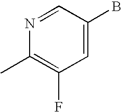 |
| 126 | Cl | CH₂CH₃ | —CH(CF₃)COOCH₂CH₃ | 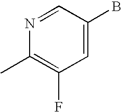 |
| 127 | Cl | CH₂CH₃ | —CH(CH₃)₂ | 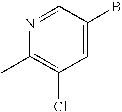 |
| 128 | Cl | CH₂CH₃ | —CH(CH₃)CH₂CH₃ | 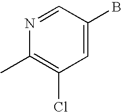 |
| 129 | Cl | CH₂CH₃ | —CH₂CH(CH₃)₂ | 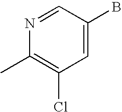 |
| 130 | Cl | CH₂CH₃ | —CH(CH₃)CH(CH₃)₂ | 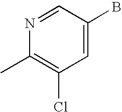 |
| 131 | Cl | CH₂CH₃ | —CH(CH₃)(CF₃) | 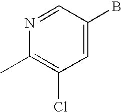 |
| 132 | Cl | CH₂CH₃ | —CH₂CF₃ | 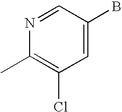 |
| 133 | Cl | CH₂CH₃ | —CH(CF₃)CH₂CH₃ | 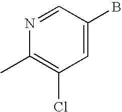 |
| 134 | Cl | CH₂CH₃ | —CH(CF₃)CH(CH₃)₂ | 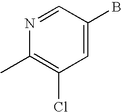 |
| 135 | Cl | CH₂CH₃ | —CH(CF₃)CH₂CH(CH₃)₂ | 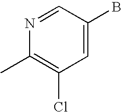 |
| 136 | Cl | CH₂CH₃ | —CH(CF₃)CH(CH₃)CH₂CH₃ | 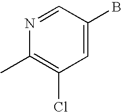 |
| 137 | Cl | CH₂CH₃ | 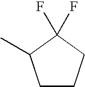 | 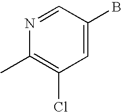 |
| 138 | Cl | CH₂CH₃ | —CF₂CF₂CF₃ | 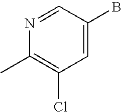 |
| 139 | Cl | CH₂CH₃ | —CH(CH[CH₃]₂)COOCH₂CH₃ | 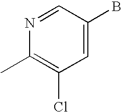 |
| 140 | Cl | CH₂CH₃ | —CH(CF₃)COOCH₂CH₃ | 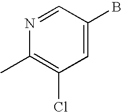 |
| 141 | Cl | CH₂C(CH₃)═CH₂ | —CH(CH₃)₂ | 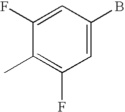 |
| 142 | Cl | CH₂C(CH₃)═CH₂ | —CH(CH₃)CH₂CH₃ | 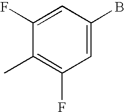 |
| 143 | Cl | CH₂C(CH₃)═CH₂ | —CH₂CH(CH₃)₂ | 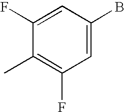 |
| 144 | Cl | CH₂C(CH₃)═CH₂ | —CH(CH₃)CH(CH₃)₂ | 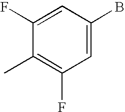 |
| 145 | Cl | CH₂C(CH₃)═CH₂ | —CH(CH₃)(CF₃) | 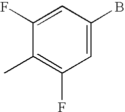 |
| 146 | Cl | CH₂C(CH₃)═CH₂ | —CH₂CF₃ | 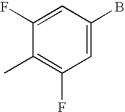 |
| 147 | Cl | CH₂C(CH₃)═CH₂ | —CH(CF₃)CH₂CH₃ | 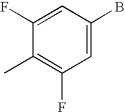 |
| 148 | Cl | CH₂C(CH₃)═CH₂ | —CH(CF₃)CH(CH₃)₂ | 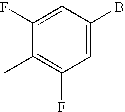 |
| 149 | Cl | CH₂C(CH₃)═CH₂ | —CH(CF₃)CH₂CH(CH₃)₂ | 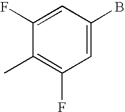 |
| 150 | Cl | CH₂C(CH₃)═CH₂ | —CH(CF₃)CH(CH₃)CH₂CH₃ | 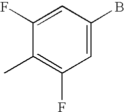 |
| 151 | Cl | CH₂C(CH₃)═CH₂ | 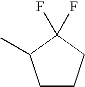 | 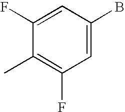 |
| 152 | Cl | CH₂C(CH₃)═CH₂ | —CF₂CF₂CF₃ | 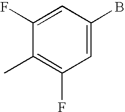 |
| 153 | Cl | CH₂C(CH₃)═CH₂ | —CH(CH[CH₃]₂)COOCH₂CH₃ | 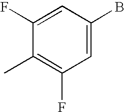 |
| 154 | Cl | CH₂C(CH₃)═CH₂ | —CH(CF₃)COOCH₂CH₃ | 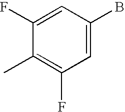 |
| 155 | Cl | CH₂C(CH₃)═CH₂ | —CH(CH₃)₂ | 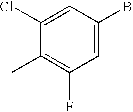 |
| 156 | Cl | CH₂C(CH₃)═CH₂ | —CH(CH₃)CH₂CH₃ | 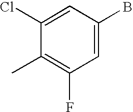 |
| 157 | Cl | CH₂C(CH₃)═CH₂ | —CH₂CH(CH₃)₂ | 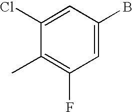 |
| 158 | Cl | CH₂C(CH₃)═CH₂ | —CH(CH₃)CH(CH₃)₂ | 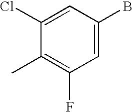 |
| 159 | Cl | CH₂C(CH₃)═CH₂ | —CH(CH₃)(CF₃) | 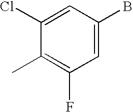 |
| 160 | Cl | CH₂C(CH₃)═CH₂ | —CH₂CF₃ | 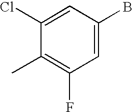 |
| 161 | Cl | CH₂C(CH₃)═CH₂ | —CH(CF₃)CH₂CH₃ | 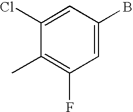 |
| 162 | Cl | CH₂C(CH₃)═CH₂ | —CH(CF₃)CH(CH₃)₂ | 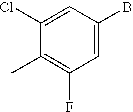 |
| 163 | Cl | CH₂C(CH₃)═CH₂ | —CH(CF₃)CH₂CH(CH₃)₂ | 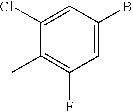 |
| 164 | Cl | CH₂C(CH₃)═CH₂ | —CH(CF₃)CH(CH₃)CH₂CH₃ | 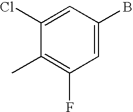 |
| 165 | Cl | CH₂C(CH₃)═CH₂ | 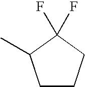 | 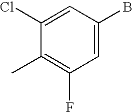 |
| 166 | Cl | CH₂C(CH₃)═CH₂ | —CF₂CF₂CF₃ | 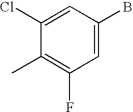 |
| 167 | Cl | CH₂C(CH₃)═CH₂ | —CH(CH[CH₃]₂)COOCH₂CH₃ | 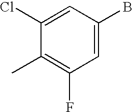 |
| 168 | Cl | CH₂C(CH₃)═CH₂ | —CH(CF₃)COOCH₂CH₃ | 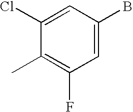 |
| 169 | Cl | CH₂C(CH₃)═CH₂ | —CH(CH₃)₂ | 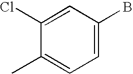 |
| 170 | Cl | CH₂C(CH₃)═CH₂ | —CH(CH₃)CH₂CH₃ | 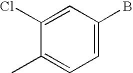 |
| 171 | Cl | CH₂C(CH₃)═CH₂ | —CH₂CH(CH₃)₂ | 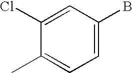 |
| 172 | Cl | CH₂C(CH₃)═CH₂ | —CH(CH₃)CH(CH₃)₂ | 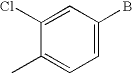 |
| 173 | Cl | CH₂C(CH₃)═CH₂ | —CH(CH₃)(CF₃) | 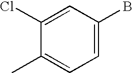 |
| 174 | Cl | CH₂C(CH₃)═CH₂ | —CH₂CF₃ | 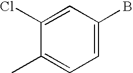 |
| 175 | Cl | CH₂C(CH₃)═CH₂ | —CH(CF₃)CH₂CH₃ | 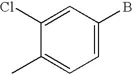 |
| 176 | Cl | CH₂C(CH₃)═CH₂ | —CH(CF₃)CH(CH₃)₂ | 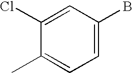 |
| 177 | Cl | CH₂C(CH₃)═CH₂ | —CH(CF₃)CH₂CH(CH₃)₂ | 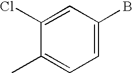 |
| 178 | Cl | CH₂C(CH₃)═CH₂ | —CH(CF₃)CH(CH₃)CH₂CH₃ | 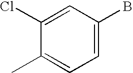 |
| 179 | Cl | CH₂C(CH₃)═CH₂ | 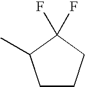 | 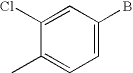 |
| 180 | Cl | CH₂C(CH₃)═CH₂ | —CF₂CF₂CF₃ | 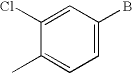 |
| 181 | Cl | CH₂C(CH₃)═CH₂ | —CH(CH[CH₃]₂)COOCH₂CH₃ | 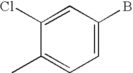 |
| 182 | Cl | CH₂C(CH₃)═CH₂ | —CH(CF₃)COOCH₂CH₃ | 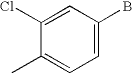 |
| 183 | Cl | CH₂C(CH₃)═CH₂ | —CH(CH₃)₂ | 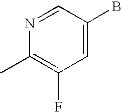 |
| 184 | Cl | CH₂C(CH₃)═CH₂ | —CH(CH₃)CH₂CH₃ | 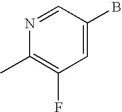 |
| 185 | Cl | CH₂C(CH₃)═CH₂ | —CH₂CH(CH₃)₂ | 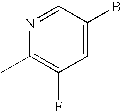 |
| 186 | Cl | CH₂C(CH₃)═CH₂ | —CH(CH₃)CH(CH₃)₂ | 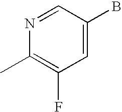 |
| 187 | Cl | CH₂C(CH₃)═CH₂ | —CH(CH₃)(CF₃) | 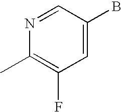 |
| 188 | Cl | CH₂C(CH₃)═CH₂ | —CH₂CF₃ | 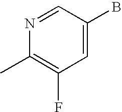 |
| 189 | Cl | CH₂C(CH₃)═CH₂ | —CH(CF₃)CH₂CH₃ | 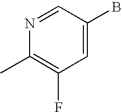 |
| 190 | Cl | CH₂C(CH₃)═CH₂ | —CH(CF₃)CH(CH₃)₂ | 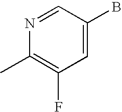 |
| 191 | Cl | CH₂C(CH₃)═CH₂ | —CH(CF₃)CH₂CH(CH₃)₂ | 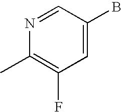 |
| 192 | Cl | CH₂C(CH₃)═CH₂ | —CH(CF₃)CH(CH₃)CH₂CH₃ | 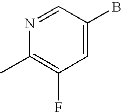 |
| 193 | Cl | CH₂C(CH₃)═CH₂ | 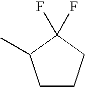 | 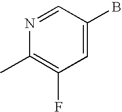 |
| 194 | Cl | CH₂C(CH₃)═CH₂ | —CF₂CF₂CF₃ | 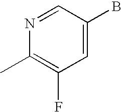 |
| 195 | Cl | CH₂C(CH₃)═CH₂ | —CH(CH[CH₃]₂)COOCH₂CH₃ | 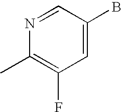 |
| 196 | Cl | CH₂C(CH₃)═CH₂ | —CH(CF₃)COOCH₂CH₃ | 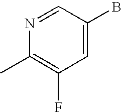 |
| 197 | Cl | CH₂C(CH₃)═CH₂ | —CH(CH₃)₂ | 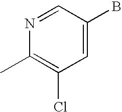 |
| 198 | Cl | CH₂C(CH₃)═CH₂ | —CH(CH₃)CH₂CH₃ | 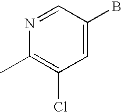 |
| 199 | Cl | CH₂C(CH₃)═CH₂ | —CH₂CH(CH₃)₂ | 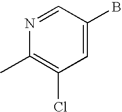 |
| 200 | Cl | CH₂C(CH₃)═CH₂ | —CH(CH₃)CH(CH₃)₂ | 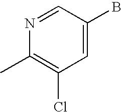 |
| 201 | Cl | CH₂C(CH₃)═CH₂ | —CH(CH₃)(CF₃) | 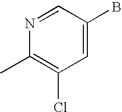 |
| 202 | Cl | CH₂C(CH₃)═CH₂ | —CH₂CF₃ | 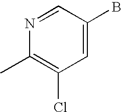 |
| 203 | Cl | CH₂C(CH₃)═CH₂ | —CH(CF₃)CH₂CH₃ | 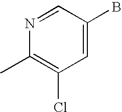 |
| 204 | Cl | CH₂C(CH₃)═CH₂ | —CH(CF₃)CH(CH₃)₂ | 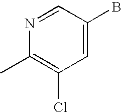 |
| 205 | Cl | CH₂C(CH₃)═CH₂ | —CH(CF₃)CH₂CH(CH₃)₂ | 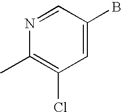 |
| 206 | Cl | CH₂C(CH₃)═CH₂ | —CH(CF₃)CH(CH₃)CH₂CH₃ | 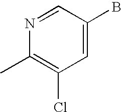 |
| 207 | Cl | CH₂C(CH₃)═CH₂ | 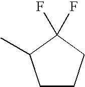 | 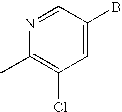 |
| 208 | Cl | CH₂C(CH₃)═CH₂ | —CF₂CF₂CF₃ | 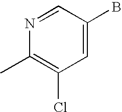 |
| 209 | Cl | CH₂C(CH₃)═CH₂ | —CH(CH[CH₃]₂)COOCH₂CH₃ | 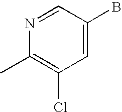 |
| 210 | Cl | CH₂C(CH₃)═CH₂ | —CH(CF₃)COOCH₂CH₃ | 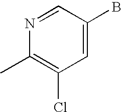 |
| 211 | F | H | —CH(CH₃)₂ | 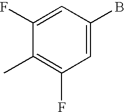 |
| 212 | F | H | —CH(CH₃)CH₂CH₃ | 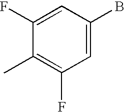 |
| 213 | F | H | —CH₂CH(CH₃)₂ | 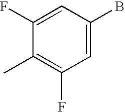 |
| 214 | F | H | —CH(CH₃)CH(CH₃)₂ | 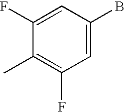 |
| 215 | F | H | —CH(CH₃)(CF₃) | 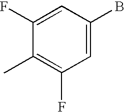 |
| 216 | F | H | —CH₂CF₃ | 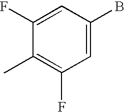 |
| 217 | F | H | —CH(CF₃)CH₂CH₃ | 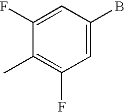 |
| 218 | F | H | —CH(CF₃)CH(CH₃)₂ | 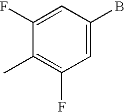 |
| 219 | F | H | —CH(CF₃)CH₂CH(CH₃)₂ | 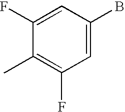 |
| 220 | F | H | —CH(CF₃)CH(CH₃)CH₂CH₃ | 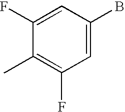 |
| 221 | F | H | 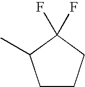 | 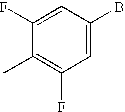 |
| 222 | F | H | —CF₂CF₂CF₃ | 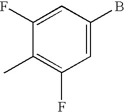 |
| 223 | F | H | —CH(CH[CH₃]₂)COOCH₂CH₃ | 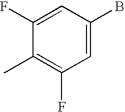 |
| 224 | F | H | —CH(CF₃)COOCH₂CH₃ | 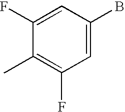 |
| 225 | F | H | —CH(CH₃)₂ | 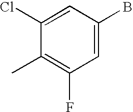 |
| 226 | F | H | —CH(CH₃)CH₂CH₃ | 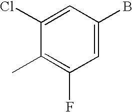 |
| 227 | F | H | —CH₂CH(CH₃)₂ | 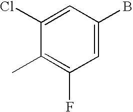 |
| 228 | F | H | —CH(CH₃)CH(CH₃)₂ | 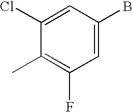 |
| 229 | F | H | —CH(CH₃)(CF₃) | 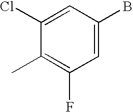 |
| 230 | F | H | —CH₂CF₃ | 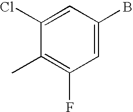 |
| 231 | F | H | —CH(CF₃)CH₂CH₃ | 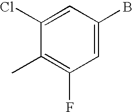 |
| 232 | F | H | —CH(CF₃)CH(CH₃)₂ | 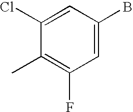 |
| 233 | F | H | —CH(CF₃)CH₂CH(CH₃)₂ | 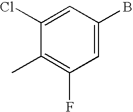 |
| 234 | F | H | —CH(CF₃)CH(CH₃)CH₂CH₃ | 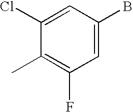 |
| 235 | F | H | 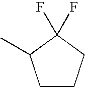 | 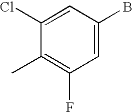 |
| 236 | F | H | —CF₂CF₂CF₃ | 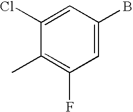 |
| 237 | F | H | —CH(CH[CH₃]₂)COOCH₂CH₃ | 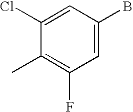 |
| 238 | F | H | —CH(CF₃)COOCH₂CH₃ | 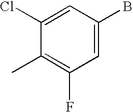 |
| 239 | F | H | —CH(CH₃)₂ | 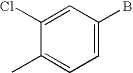 |
| 240 | F | H | —CH(CH₃)CH₂CH₃ | 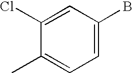 |
| 241 | F | H | —CH₂CH(CH₃)₂ | 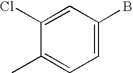 |
| 242 | F | H | —CH(CH₃)CH(CH₃)₂ | 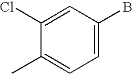 |
| 243 | F | H | —CH(CH₃)(CF₃) | 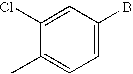 |
| 244 | F | H | —CH₂CF₃ | 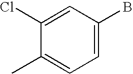 |
| 245 | F | H | —CH(CF₃)CH₂CH₃ | 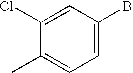 |
| 246 | F | H | —CH(CF₃)CH(CH₃)₂ | 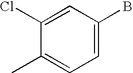 |
| 247 | F | H | —CH(CF₃)CH₂CH(CH₃)₂ | 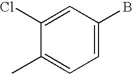 |
| 248 | F | H | —CH(CF₃)CH(CH₃)CH₂CH₃ | 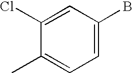 |
| 249 | F | H | 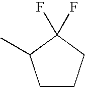 | 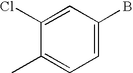 |
| 250 | F | H | —CF₂CF₂CF₃ | 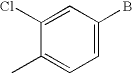 |
| 251 | F | H | —CH(CH[CH₃]₂)COOCH₂CH₃ | 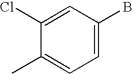 |
| 252 | F | H | —CH(CF₃)COOCH₂CH₃ | 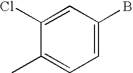 |
| 253 | F | H | —CH(CH₃)₂ | 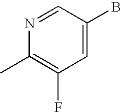 |
| 254 | F | H | —CH(CH₃)CH₂CH₃ | 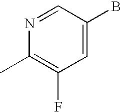 |
| 255 | F | H | —CH₂CH(CH₃)₂ | 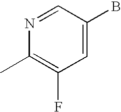 |
| 256 | F | H | —CH(CH₃)CH(CH₃)₂ | 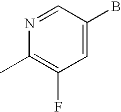 |
| 257 | F | H | —CH(CH₃)(CF₃) | 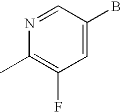 |
| 258 | F | H | —CH₂CF₃ | 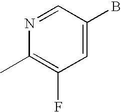 |
| 259 | F | H | —CH(CF₃)CH₂CH₃ | 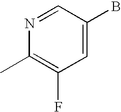 |
| 260 | F | H | —CH(CF₃)CH(CH₃)₂ | 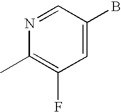 |
| 261 | F | H | —CH(CF₃)CH₂CH(CH₃)₂ | 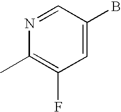 |
| 262 | F | H | —CH(CF₃)CH(CH₃)CH₂CH₃ | 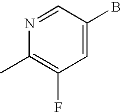 |
| 263 | F | H | 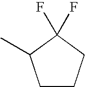 | 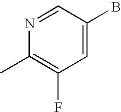 |
| 264 | F | H | —CF₂CF₂CF₃ | 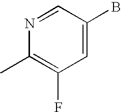 |
| 265 | F | H | —CH(CH[CH₃]₂)COOCH₂CH₃ | 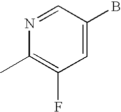 |
| 266 | F | H | —CH(CF₃)COOCH₂CH₃ | 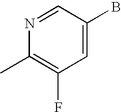 |
| 267 | F | H | —CH(CH₃)₂ | 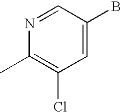 |
| 268 | F | H | —CH(CH₃)CH₂CH₃ | 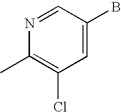 |
| 269 | F | H | —CH₂CH(CH₃)₂ | 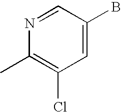 |
| 270 | F | H | —CH(CH₃)CH(CH₃)₂ | 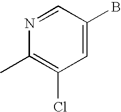 |
| 271 | F | H | —CH(CH₃)(CF₃) | 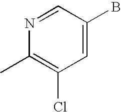 |
| 272 | F | H | —CH₂CF₃ | 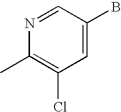 |
| 273 | F | H | —CH(CF₃)CH₂CH₃ | 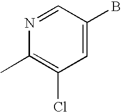 |
| 274 | F | H | —CH(CF₃)CH(CH₃)₂ | 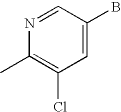 |
| 275 | F | H | —CH(CF₃)CH₂CH(CH₃)₂ | 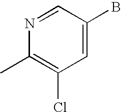 |
| 276 | F | H | —CH(CF₃)CH(CH₃)CH₂CH₃ | 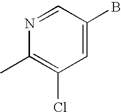 |
| 277 | F | H | 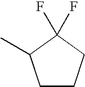 | 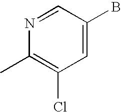 |
| 278 | F | H | —CF₂CF₂CF₃ | 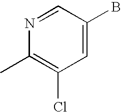 |
| 279 | F | H | —CH(CH[CH₃]₂)COOCH₂CH₃ | 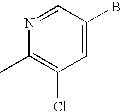 |
| 280 | F | H | —CH(CF₃)COOCH₂CH₃ | 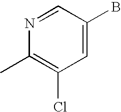 |
| 281 | F | CH₂CH₃ | —CH(CH₃)₂ | 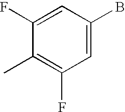 |
| 282 | F | CH₂CH₃ | —CH(CH₃)CH₂CH₃ | 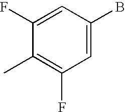 |
| 283 | F | CH₂CH₃ | —CH₂CH(CH₃)₂ | 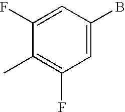 |
| 284 | F | CH₂CH₃ | —CH(CH₃)CH(CH₃)₂ | 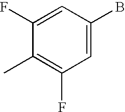 |
| 285 | F | CH₂CH₃ | —CH(CH₃)(CF₃) | 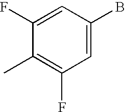 |
| 286 | F | CH₂CH₃ | —CH₂CF₃ | 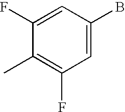 |
| 287 | F | CH₂CH₃ | —CH(CF₃)CH₂CH₃ | 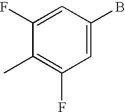 |
| 288 | F | CH₂CH₃ | —CH(CF₃)CH(CH₃)₂ | 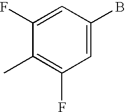 |
| 289 | F | CH₂CH₃ | —CH(CF₃)CH₂CH(CH₃)₂ | 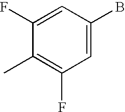 |
| 290 | F | CH₂CH₃ | —CH(CF₃)CH(CH₃)CH₂CH₃ | 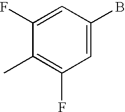 |
| 291 | F | CH₂CH₃ | 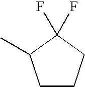 | 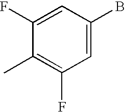 |
| 292 | F | CH₂CH₃ | —CF₂CF₂CF₃ | 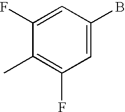 |
| 293 | F | CH₂CH₃ | —CH(CH[CH₃]₂)COOCH₂CH₃ | 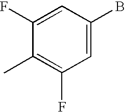 |
| 294 | F | CH₂CH₃ | —CH(CF₃)COOCH₂CH₃ | 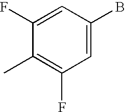 |
| 295 | F | CH₂CH₃ | —CH(CH₃)₂ | 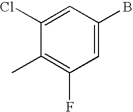 |
| 296 | F | CH₂CH₃ | —CH(CH₃)CH₂CH₃ | 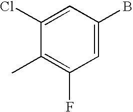 |
| 297 | F | CH₂CH₃ | —CH₂CH(CH₃)₂ | 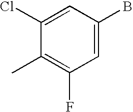 |
| 298 | F | CH₂CH₃ | —CH(CH₃)CH(CH₃)₂ | 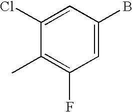 |
| 299 | F | CH₂CH₃ | —CH(CH₃)(CF₃) | 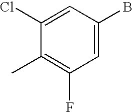 |
| 300 | F | CH₂CH₃ | —CH₂CF₃ | 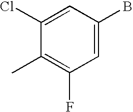 |
| 301 | F | CH₂CH₃ | —CH(CF₃)CH₂CH₃ | 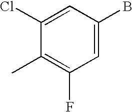 |
| 302 | F | CH₂CH₃ | —CH(CF₃)CH(CH₃)₂ | 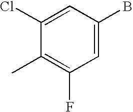 |
| 303 | F | CH₂CH₃ | —CH(CF₃)CH₂CH(CH₃)₂ | 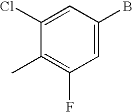 |
| 304 | F | CH₂CH₃ | —CH(CF₃)CH(CH₃)CH₂CH₃ | 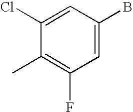 |
| 305 | F | CH₂CH₃ | 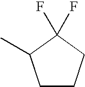 | 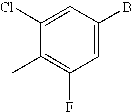 |
| 306 | F | CH₂CH₃ | —CF₂CF₂CF₃ | 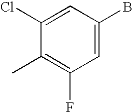 |
| 307 | F | CH₂CH₃ | —CH(CH[CH₃]₂)COOCH₂CH₃ | 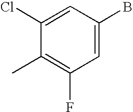 |
| 308 | F | CH₂CH₃ | —CH(CF₃)COOCH₂CH₃ | 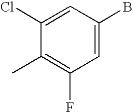 |
| 309 | F | CH₂CH₃ | —CH(CH₃)₂ | 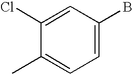 |
| 310 | F | CH₂CH₃ | —CH(CH₃)CH₂CH₃ | 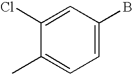 |
| 311 | F | CH₂CH₃ | —CH₂CH(CH₃)₂ | 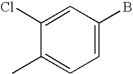 |
| 312 | F | CH₂CH₃ | —CH(CH₃)CH(CH₃)₂ | 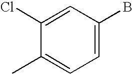 |
| 313 | F | CH₂CH₃ | —CH(CH₃)(CF₃) | 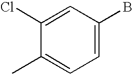 |
| 314 | F | CH₂CH₃ | —CH₂CF₃ | 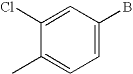 |
| 315 | F | CH₂CH₃ | —CH(CF₃)CH₂CH₃ | 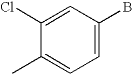 |
| 316 | F | CH₂CH₃ | —CH(CF₃)CH(CH₃)₂ | 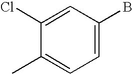 |
| 317 | F | CH₂CH₃ | —CH(CF₃)CH₂CH(CH₃)₂ | 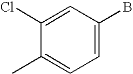 |
| 318 | F | CH₂CH₃ | —CH(CF₃)CH(CH₃)CH₂CH₃ | 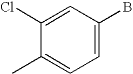 |
| 319 | F | CH₂CH₃ | 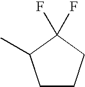 | 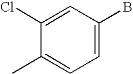 |
| 320 | F | CH₂CH₃ | —CF₂CF₂CF₃ | 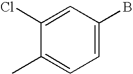 |
| 321 | F | CH₂CH₃ | —CH(CH[CH₃]₂)COOCH₂CH₃ | 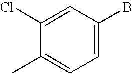 |
| 322 | F | CH₂CH₃ | —CH(CF₃)COOCH₂CH₃ | 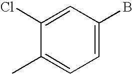 |
| 323 | F | CH₂CH₃ | —CH(CH₃)₂ | 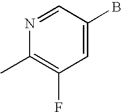 |
| 324 | F | CH₂CH₃ | —CH(CH₃)CH₂CH₃ | 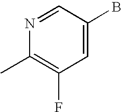 |
| 325 | F | CH₂CH₃ | —CH₂CH(CH₃)₂ | 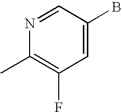 |
| 326 | F | CH₂CH₃ | —CH(CH₃)CH(CH₃)₂ | 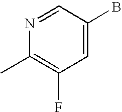 |
| 327 | F | CH₂CH₃ | —CH(CH₃)(CF₃) | 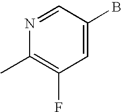 |
| 328 | F | CH₂CH₃ | —CH₂CF₃ | 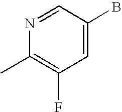 |
| 329 | F | CH₂CH₃ | —CH(CF₃)CH₂CH₃ | 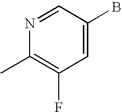 |
| 330 | F | CH₂CH₃ | —CH(CF₃)CH(CH₃)₂ | 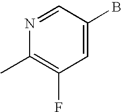 |
| 331 | F | CH₂CH₃ | —CH(CF₃)CH₂CH(CH₃)₂ | 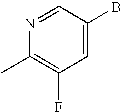 |
| 332 | F | CH₂CH₃ | —CH(CF₃)CH(CH₃)CH₂CH₃ | 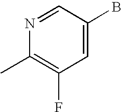 |
| 333 | F | CH₂CH₃ | 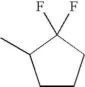 | 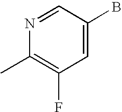 |
| 334 | F | CH₂CH₃ | —CF₂CF₂CF₃ | 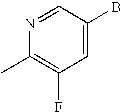 |
| 335 | F | CH₂CH₃ | —CH(CH[CH₃]₂)COOCH₂CH₃ | 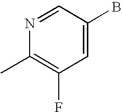 |
| 336 | F | CH₂CH₃ | —CH(CF₃)COOCH₂CH₃ | 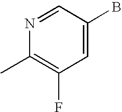 |
| 337 | F | CH₂CH₃ | —CH(CH₃)₂ | 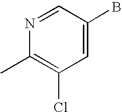 |
| 338 | F | CH₂CH₃ | —CH(CH₃)CH₂CH₃ | 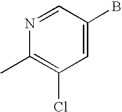 |
| 339 | F | CH₂CH₃ | —CH₂CH(CH₃)₂ | 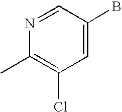 |
| 340 | F | CH₂CH₃ | —CH(CH₃)CH(CH₃)₂ | 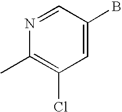 |
| 341 | F | CH₂CH₃ | —CH(CH₃)(CF₃) | 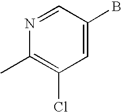 |
| 342 | F | CH₂CH₃ | —CH₂CF₃ | 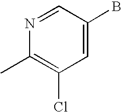 |
| 343 | F | CH₂CH₃ | —CH(CF₃)CH₂CH₃ | 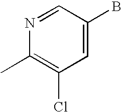 |
| 344 | F | CH₂CH₃ | —CH(CF₃)CH(CH₃)₂ | 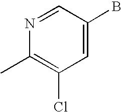 |
| 345 | F | CH₂CH₃ | —CH(CF₃)CH₂CH(CH₃)₂ | 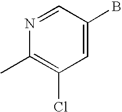 |
| 346 | F | CH₂CH₃ | —CH(CF₃)CH(CH₃)CH₂CH₃ | 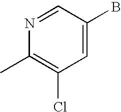 |
| 347 | F | CH₂CH₃ | 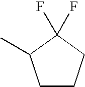 | 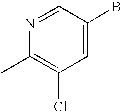 |
| 348 | F | CH₂CH₃ | —CF₂CF₂CF₃ | 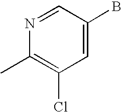 |
| 349 | F | CH₂CH₃ | —CH(CH[CH₃]₂)COOCH₂CH₃ | 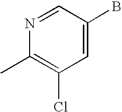 |
| 350 | F | CH₂CH₃ | —CH(CF₃)COOCH₂CH₃ | 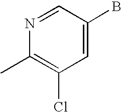 |
| 351 | F | CH₂C(CH₃)═CH₂ | —CH(CH₃)₂ | 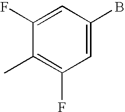 |
| 352 | F | CH₂C(CH₃)═CH₂ | —CH(CH₃)CH₂CH₃ | 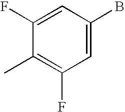 |
| 353 | F | CH₂C(CH₃)═CH₂ | —CH₂CH(CH₃)₂ | 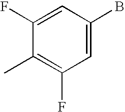 |
| 354 | F | CH₂C(CH₃)═CH₂ | —CH(CH₃)CH(CH₃)₂ | 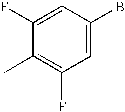 |
| 355 | F | CH₂C(CH₃)═CH₂ | —CH(CH₃)(CF₃) | 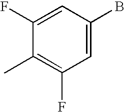 |
| 356 | F | CH₂C(CH₃)═CH₂ | —CH₂CF₃ | 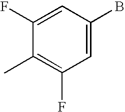 |
| 357 | F | CH₂C(CH₃)═CH₂ | —CH(CF₃)CH₂CH₃ | 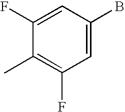 |
| 358 | F | CH₂C(CH₃)═CH₂ | —CH(CF₃)CH(CH₃)₂ | 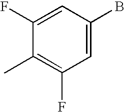 |
| 359 | F | CH₂C(CH₃)═CH₂ | —CH(CF₃)CH₂CH(CH₃)₂ | 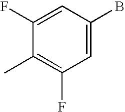 |
| 360 | F | CH₂C(CH₃)═CH₂ | —CH(CF₃)CH(CH₃)CH₂CH₃ | 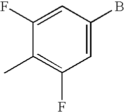 |
| 361 | F | CH₂C(CH₃)═CH₂ | 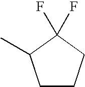 | 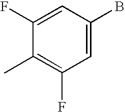 |
| 362 | F | CH₂C(CH₃)═CH₂ | —CF₂CF₂CF₃ | 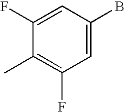 |
| 363 | F | CH₂C(CH₃)═CH₂ | —CH(CH[CH₃]₂)COOCH₂CH₃ | 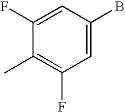 |
| 364 | F | CH₂C(CH₃)═CH₂ | —CH(CF₃)COOCH₂CH₃ | 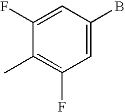 |
| 365 | F | CH₂C(CH₃)═CH₂ | —CH(CH₃)₂ | 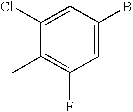 |
| 366 | F | CH₂C(CH₃)═CH₂ | —CH(CH₃)CH₂CH₃ | 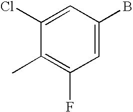 |
| 367 | F | CH₂C(CH₃)═CH₂ | —CH₂CH(CH₃)₂ | 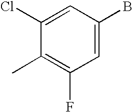 |
| 368 | F | CH₂C(CH₃)═CH₂ | —CH(CH₃)CH(CH₃)₂ | 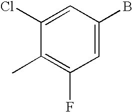 |
| 369 | F | CH₂C(CH₃)═CH₂ | —CH(CH₃)(CF₃) | 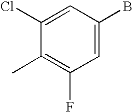 |
| 370 | F | CH₂C(CH₃)═CH₂ | —CH₂CF₃ | 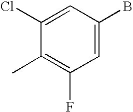 |
| 371 | F | CH₂C(CH₃)═CH₂ | —CH(CF₃)CH₂CH₃ | 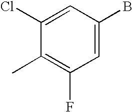 |
| 372 | F | CH₂C(CH₃)═CH₂ | —CH(CF₃)CH(CH₃)₂ | 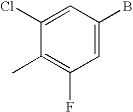 |
| 373 | F | CH₂C(CH₃)═CH₂ | —CH(CF₃)CH₂CH(CH₃)₂ | 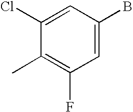 |
| 374 | F | CH₂C(CH₃)═CH₂ | —CH(CF₃)CH(CH₃)CH₂CH₃ | 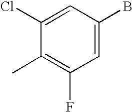 |
| 375 | F | CH₂C(CH₃)═CH₂ | 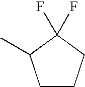 | 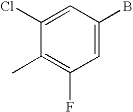 |
| 376 | F | CH₂C(CH₃)═CH₂ | —CF₂CF₂CF₃ | 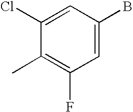 |
| 377 | F | CH₂C(CH₃)═CH₂ | —CH(CH[CH₃]₂)COOCH₂CH₃ | 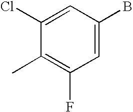 |
| 378 | F | CH₂C(CH₃)═CH₂ | —CH(CF₃)COOCH₂CH₃ | 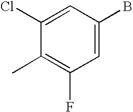 |
| 379 | F | CH₂C(CH₃)═CH₂ | —CH(CH₃)₂ | 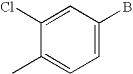 |
| 380 | F | CH₂C(CH₃)═CH₂ | —CH(CH₃)CH₂CH₃ | 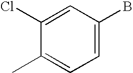 |
| 381 | F | CH₂C(CH₃)═CH₂ | —CH₂CH(CH₃)₂ | 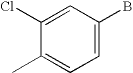 |
| 382 | F | CH₂C(CH₃)═CH₂ | —CH(CH₃)CH(CH₃)₂ | 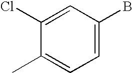 |
| 383 | F | CH₂C(CH₃)═CH₂ | —CH(CH₃)(CF₃) | 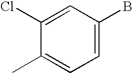 |
| 384 | F | CH₂C(CH₃)═CH₂ | —CH₂CF₃ | 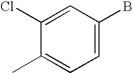 |
| 385 | F | CH₂C(CH₃)═CH₂ | —CH(CF₃)CH₂CH₃ | 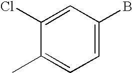 |
| 386 | F | CH₂C(CH₃)═CH₂ | —CH(CF₃)CH(CH₃)₂ | 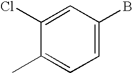 |
| 387 | F | CH₂C(CH₃)═CH₂ | —CH(CF₃)CH₂CH(CH₃)₂ | 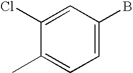 |
| 388 | F | CH₂C(CH₃)═CH₂ | —CH(CF₃)CH(CH₃)CH₂CH₃ | 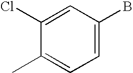 |
| 389 | F | CH₂C(CH₃)═CH₂ | 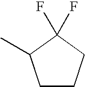 | 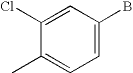 |
| 390 | F | CH₂C(CH₃)═CH₂ | —CF₂CF₂CF₃ | 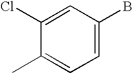 |
| 391 | F | CH₂C(CH₃)═CH₂ | —CH(CH[CH₃]₂)COOCH₂CH₃ | 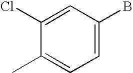 |
| 392 | F | CH₂C(CH₃)═CH₂ | —CH(CF₃)COOCH₂CH₃ | 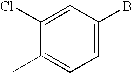 |
| 393 | F | CH₂C(CH₃)═CH₂ | —CH(CH₃)₂ | 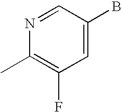 |
| 394 | F | CH₂C(CH₃)═CH₂ | —CH(CH₃)CH₂CH₃ | 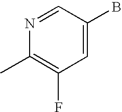 |
| 395 | F | CH₂C(CH₃)═CH₂ | —CH₂CH(CH₃)₂ | 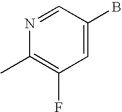 |
| 396 | F | CH₂C(CH₃)═CH₂ | —CH(CH₃)CH(CH₃)₂ | 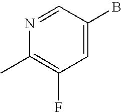 |
| 397 | F | CH₂C(CH₃)═CH₂ | —CH(CH₃)(CF₃) | 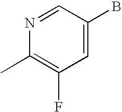 |
| 398 | F | CH₂C(CH₃)═CH₂ | —CH₂CF₃ | 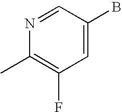 |
| 399 | F | CH₂C(CH₃)═CH₂ | —CH(CF₃)CH₂CH₃ | 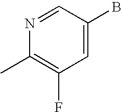 |
| 400 | F | CH₂C(CH₃)═CH₂ | —CH(CF₃)CH(CH₃)₂ | 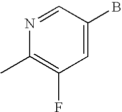 |
| 401 | F | CH₂C(CH₃)═CH₂ | —CH(CF₃)CH₂CH(CH₃)₂ | 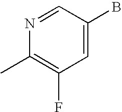 |
| 402 | F | CH₂C(CH₃)═CH₂ | —CH(CF₃)CH(CH₃)CH₂CH₃ | 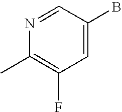 |
| 403 | F | CH₂C(CH₃)═CH₂ | 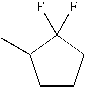 | 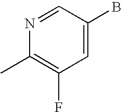 |
| 404 | F | CH₂C(CH₃)═CH₂ | —CF₂CF₂CF₃ | 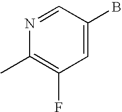 |
| 405 | F | CH₂C(CH₃)═CH₂ | —CH(CH[CH₃]₂)COOCH₂CH₃ | 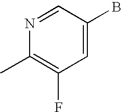 |
| 406 | F | CH₂C(CH₃)═CH₂ | —CH(CF₃)COOCH₂CH₃ | 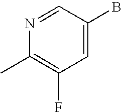 |
| 407 | F | CH₂C(CH₃)═CH₂ | —CH(CH₃)₂ | 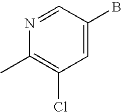 |
| 408 | F | CH₂C(CH₃)═CH₂ | —CH(CH₃)CH₂CH₃ | 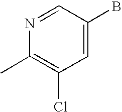 |
| 409 | F | CH₂C(CH₃)═CH₂ | —CH₂CH(CH₃)₂ | 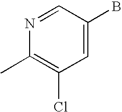 |
| 410 | F | CH₂C(CH₃)═CH₂ | —CH(CH₃)CH(CH₃)₂ | 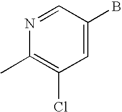 |
| 411 | F | CH₂C(CH₃)═CH₂ | —CH(CH₃)(CF₃) | 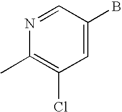 |
| 412 | F | CH₂C(CH₃)═CH₂ | —CH₂CF₃ | 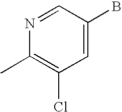 |
| 413 | F | CH₂C(CH₃)═CH₂ | —CH(CF₃)CH₂CH₃ | 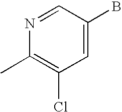 |
| 414 | F | CH₂C(CH₃)═CH₂ | —CH(CF₃)CH(CH₃)₂ | 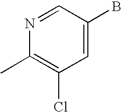 |
| 415 | F | CH₂C(CH₃)═CH₂ | —CH(CF₃)CH₂CH(CH₃)₂ | 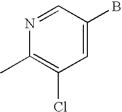 |
| 416 | F | CH₂C(CH₃)═CH₂ | —CH(CF₃)CH(CH₃)CH₂CH₃ | 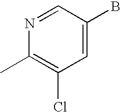 |
| 417 | F | CH₂C(CH₃)═CH₂ | 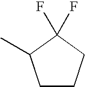 | 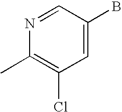 |
| 418 | F | CH₂C(CH₃)═CH₂ | —CF₂CF₂CF₃ | 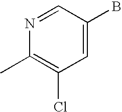 |
| 419 | F | CH₂C(CH₃)═CH₂ | —CH(CH[CH₃]₂)COOCH₂CH₃ | 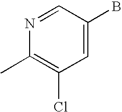 |
| 420 | F | CH₂C(CH₃)═CH₂ | —CH(CF₃)COOCH₂CH₃ | 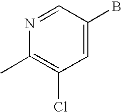 |
| 421 | Cl | H | CH(CH[CH₃]₂)COOCH₃ | 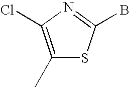 |
| 422 | Cl | CH₂CH₃ | —CH₂CF₃ | 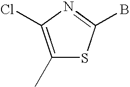 |
| 423 | Cl | CH₂C(CH₃)═CH₂ | —CH(CF₃)CH(CH₃)CH₂CH₃ | 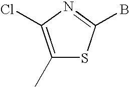 |
| 424 | F | H | CH(CH[CH₃]₂)COOCH₃ | 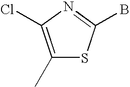 |
| 425 | F | CH₂CH₃ | —CH₂CF₃ | 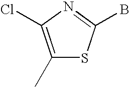 |
| 426 | F | CH₂C(CH₃)═CH₂ | —CH(CF₃)CH(CH₃)CH₂CH₃ | 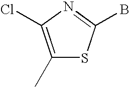 |
| 427 | Cl | H | CH(CH[CH₃]₂)COOCH₃ | 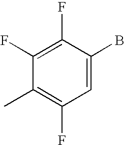 |
| 428 | Cl | CH₂CH₃ | —CH₂CF₃ | 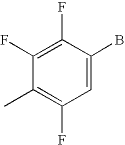 |
| 429 | Cl | CH₂C(CH₃)═CH₂ | —CH(CF₃)CH(CH₃)CH₂CH₃ | 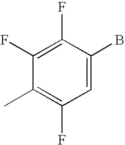 |
| 430 | F | H | CH(CH[CH₃]₂)COOCH₃ | 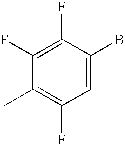 |
| 431 | F | CH₂CH₃ | —CH₂CF₃ | 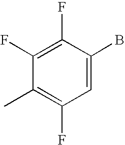 |
| 432 | F | CH₂C(CH₃)═CH₂ | —CH(CF₃)CH(CH₃)CH₂CH₃ | 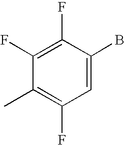 |
| 433 | Cl | H | CH(CH[CH₃]₂)COOCH₃ | 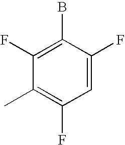 |
| 434 | Cl | CH₂CH₃ | —CH₂CF₃ | 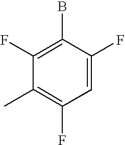 |
| 435 | Cl | CH₂C(CH₃)═CH₂ | —CH(CF₃)CH(CH₃)CH₂CH₃ | 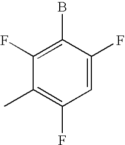 |
| 436 | F | H | CH(CH[CH₃]₂)COOCH₃ | 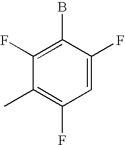 |
| 437 | F | CH₂CH₃ | —CH₂CF₃ | 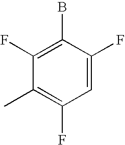 |
| 438 | F | CH₂C(CH₃)═CH₂ | —CH(CF₃)CH(CH₃)CH₂CH₃ | 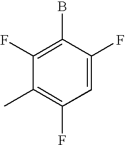 |
| 439 | Cl | H | CH(CH[CH₃]₂)COOCH₃ | 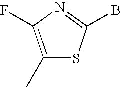 |
| 440 | Cl | CH₂CH₃ | —CH₂CF₃ | 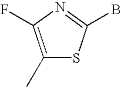 |
| 441 | Cl | CH₂C(CH₃)═CH₂ | —CH(CF₃)CH(CH₃)CH₂CH₃ | 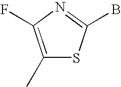 |
| 442 | F | H | CH(CH[CH₃]₂)COOCH₃ | 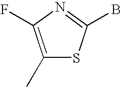 |
| 443 | F | CH₂CH₃ | —CH₂CF₃ | 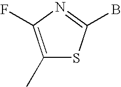 |
| 444 | F | CH₂C(CH₃)═CH₂ | —CH(CF₃)CH(CH₃)CH₂CH₃ | 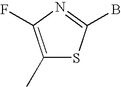 |
| 445 | CN | —CH═CH₂ | 3-Pyridyl | 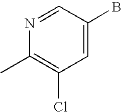 |
| 446 | CF₃ | NH₂ | Phenyl | 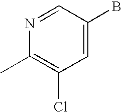 |
| 447 | Br | —NH-Phenyl | cyclohexyl | 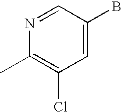 |
| 448 | CN | —CH═CH₂ | Phenyl | 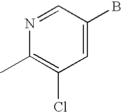 |
| 449 | CF₃ | NH₂ | 3-Pyridyl | 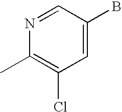 |
| 450 | Br | —NH-phenyl | 3-Pyridyl | 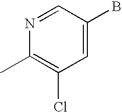 |
| 451 | CN | —CH═CH₂ | 3-Pyridyl | 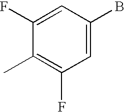 |
| 452 | CF₃ | NH₂ | Phenyl | 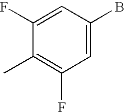 |
| 453 | Br | —NH-Phenyl | cyclohexyl | 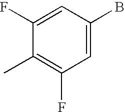 |

TABLE 2
Table 2 consists of 453 compounds of the general formula (2), where W
and Z are N, X and Y are CH, B is phenyl, and the values of R, R³, R⁴
and R²⁰ are as listed in Table 1.

TABLE 3
Table 3 consists of 453 compounds of the general formula (2), where W
and Z are N, X and Y are CH, B is 4-fluorophenyl, and the values of R,
R³, R⁴ and R²⁰ are as listed in Table 1.

TABLE 4
Table 4 consists of 453 compounds of the general formula (2), where W
and Z are N, X and Y are CH, B is 4-chlorophenyl, and the values of R,
R³, R⁴ and R²⁰ are as listed in Table 1.

TABLE 5
Table 5 consists of 453 compounds of the general formula (2), where W
and Z are N, X and Y are CH, B is 5-fluoro-2-pyridyl, and the values
of R, R³, R⁴ and R²⁰ are as listed in Table 1.

TABLE 6
Table 6 consists of 453 compounds of the general formula (2), where W
and Z are N, X and Y are CH, B is 6-fluoro-3-pyridyl, and the values
of R, R³, R⁴ and R²⁰ are as listed in Table 1.

TABLE 7
Table 7 consists of 453 compounds of the general formula (2), where W
and Z are N, X and Y are CH, B is 2-phenylethenyl, and the values of
R, R³, R⁴ and R²⁰ are as listed in Table 1.

TABLE 8
Table 8 consists of 453 compounds of the general formula (2), where W
and Z are N, X and Y are CH, B is 2-(4-fluorophenyl)ethenyl, and the
values of R, R³, R⁴ and R²⁰ are as listed in Table 1.

TABLE 9
Table 9 consists of 453 compounds of the general formula (2), where W
and Z are N, X and Y are CH, B is (4-methylphenyl)ethynyl, and the
values of R, R³, R⁴ and R²⁰ are as listed in Table 1.

TABLE 10
Table 10 consists of 453 compounds of the general formula (2), where W
and Z are N, X and Y are CH, B is (4-fluorophenyl)ethynyl, and the
values of R, R³, R⁴ and R²⁰ are as listed in Table 1.

TABLE 11
Table 11 consists of 453 compounds of the general formula (2), where W
and Z are N, X and Y are CH, B is 4-fluorophenoxy, and the values of
R, R³, R⁴ and R²⁰ are as listed in Table 1.

TABLE 12
Table 12 consists of 453 compounds of the general formula (2), where W
and Z are N, X and Y are CH, B is phenylthio, and the values of R,
R³, R⁴ and R²⁰ are as listed in Table 1.

TABLE 13
Table 13 consists of 453 compounds of the general formula (2), where W
and Z are N, X and Y are CH, B is phenylsulphinyl, and the values of
R, R³, R⁴ and R²⁰ are as listed in Table 1.

TABLE 14
Table 14 consists of 453 compounds of the general formula (2), where W
and Z are N, X and Y are CH, B is phenylsulphonyl, and the values of
R, R³, R⁴ and R²⁰ are as listed in Table 1.

TABLE 15
Table 15 consists of 453 compounds of the general formula (2), where W,
Y and Z are N, X is CH, B is phenyl, and the values of R, R³, R⁴ and
R²⁰ are as listed in Table 1.

TABLE 16
Table 16 consists of 453 compounds of the general formula (2), where W,
Y and Z are N, X is CH, B is 4-fluorophenyl, and the values of R, R³,
R⁴ and R²⁰ are as listed in Table 1.

TABLE 17
Table 17 consists of 453 compounds of the general formula (2), where W,
Y and Z are N, X is CH, B is 4-chlorophenyl, and the values of R, R³,
R⁴ and R²⁰ are as listed in Table 1.

TABLE 18
Table 18 consists of 453 compounds of the general formula (2), where W,
Y and Z are N, X is CH, B is 5-fluoro-2-pyridyl, and the values of R,
R³, R⁴ and R²⁰ are as listed in Table 1.

TABLE 19
Table 19 consists of 453 compounds of the general formula (2), where W,
Y and Z are N, X is CH, B is 6-fluoro-3-pyridyl, and the values of R,
R³, R⁴ and R²⁰ are as listed in Table 1.

TABLE 20
Table 20 consists of 453 compounds of the general formula (2), where W,
Y and Z are N, X is CH, B is 2-phenylethenyl, and the values of R, R³,
R⁴ and R²⁰ are as listed in Table 1.

TABLE 21
Table 21 consists of 453 compounds of the general formula (2), where W,
Y and Z are N, X is CH, B is 2-(4-fluorophenyl)ethenyl, and the values
of R, R³, R⁴ and R²⁰ are as listed in Table 1.

TABLE 22
Table 22 consists of 453 compounds of the general formula (2), where W,
Y and Z are N, X is CH, B is (4-methylphenyl)ethynyl, and the values
of R, R³, R⁴ and R²⁰ are as listed in Table 1.

TABLE 23
Table 23 consists of 453 compounds of the general formula (2), where W,
Y and Z are N, X is CH, B is (4-fluorophenyl)ethynyl, and the values
of R, R³, R⁴ and R²⁰ are as listed in Table 1.

TABLE 24
Table 24 consists of 453 compounds of the general formula (2), where W,
Y and Z are N, X is CH, B is 4-fluorophenoxy, and the values of R, R³,
R⁴ and R²⁰ are as listed in Table 1.

TABLE 25
Table 25 consists of 453 compounds of the general formula (2), where W,
Y and Z are N, X is CH, B is phenylthio, and the values of R, R³, R⁴
and R²⁰ are as listed in Table 1.

TABLE 26
Table 26 consists of 453 compounds of the general formula (2), where W,
Y and Z are N, X is CH, B is phenylsulphinyl, and the values of R, R³,
R⁴ and R²⁰ are as listed in Table 1.

TABLE 27
Table 27 consists of 453 compounds of the general formula (2), where W,
Y and Z are N, X is CH, B is phenylsulphonyl, and the values of R, R³,
R⁴ and R²⁰ are as listed in Table 1.

Compounds of formula (7) or (8), which are examples of compounds of general formula (1) where one of R and R² is NR³R⁴, can be made as shown in Scheme 1, in which W, X, Y, Z, R¹, R³ and R⁴ have the meanings given above and R⁷ is C₁₋₄ alkyl.

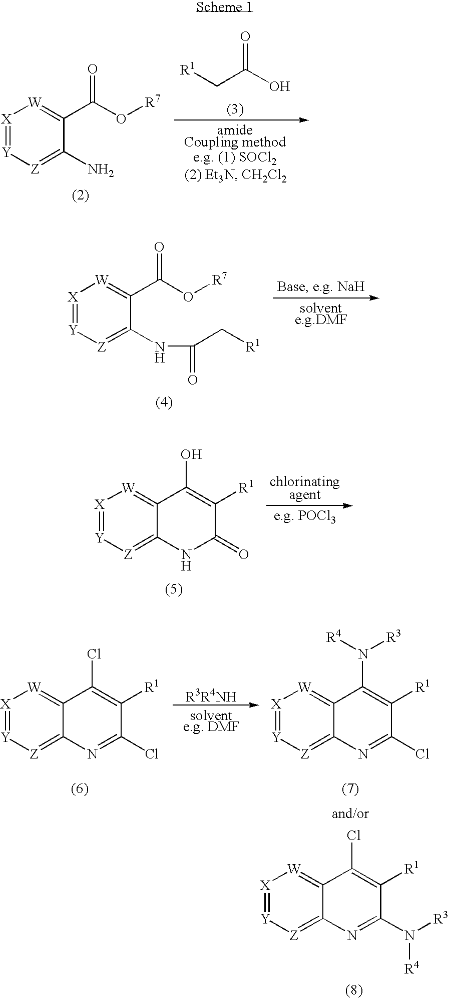

Compounds of general formula (4) can be prepared from compounds of general formula (2), which are either commercially available or made by methods known in the literature, by reaction with acids of general formula (3), using standard coupling methods, for example by conversion to the acid chloride using a chlorinating agent such as thionyl chloride, followed by reaction of the resultant acid chloride optionally in the presence of a base such as triethylamine, in a suitable solvent such as dichloromethane or toluene. Compounds of general formula (5) can be prepared by treating compounds of general formula (4) with a base such as sodium hydride, optionally in the presence of a Lewis acid such as magnesium oxide, in a suitable solvent such as N,N-dimethylformamide (DMF) or toluene, at between room temperature and 150° C., but preferably at 60-90° C. Compounds of general formula (6) can be prepared by reaction of compounds of general formula (5) with a chlorination reagent such as phosphorus oxychloride, either neat or in a suitable solvent such as toluene, at between 50 and 150° C., but preferably between 80 and 110° C., or in a microwave reactor at between 150 and 300° C., but preferably between 200 and 250° C. Compounds of formula (7) and (8) can be prepared by reaction of compounds of general formula (6) with an amine R³R⁴NH, either neat, or in a suitable solvent such as DMF, between room temperature and 150° C., but preferably between 50 and 80° C. If compounds (7) and (8) are produced as a mixture they can be separated by suitable means such as crystallisation or chromatography under normal or reverse phase conditions.

Compounds of formula (15) and (16), which are examples of compounds of general formula (1) can be made as shown in Scheme 2, where Hal is a halogen such as bromine or iodine. Compounds of formula (10), can be made by reaction of compounds of formula (9), which are examples of compounds of formula (5) in Scheme 1, by reaction with a compound B-D, where B is a substituent as defined above for R¹, and D is a metallic group such as a boronic acid B(OH)₂, or a tri(C₁₋₄) alkyl tin, in a cross-coupling reaction in the presence of a palladium catalyst for example PdP(Ph₃)₄ or Pd₂(dba)₃, a ligand for example PPh₃ or P(t-Bu)₃, a base for example K₂CO₃ or CsF, in a suitable solvent such as toluene or ethanol, at room temperature to reflux, but preferably at between 50 and 100° C.

Compounds of formula (11) can be formed by reaction of compounds of formula (9) with a chlorination reagent such as phosphorus oxychloride, either neat or in a suitable solvent such as toluene or dichlormethane, at between 50 and 150° C., but preferably between 60 and 110° C.

Compounds of formula (12) can be made either by cross-coupling of compounds of formula (11) using conditions for converting (9) to (10), or by chlorination of compounds of formula (10) using conditions for converting (9) to (11).

Compounds of formula (13) and (14) can be prepared by reacting compounds of formula (11) with an amine R³R⁴NH, either neat or in a suitable solvent such as DMF between room temperature, but preferably between 50 and 80° C. If compounds (13) and (14) are produced as a mixture they can be separated by suitable means such as crystallisation or chromatography under normal or reverse phase conditions.

Compounds of formula (15) and (16) can be prepared by reacting compounds of formula (12) with an amine R³R⁴NH, either neat or in a suitable solvent such as DMF between room temperature, but preferably between 50 and 80° C. If compounds (15) and (16) are produced as a mixture they can be separated by suitable means such as crystallisation or chromatography under normal or reverse phase conditions. Compounds of formula (15) and (16) can also be prepared individually from compounds of formula (13) and (14) respectively by cross-coupling using conditions for converting (9) to (10).

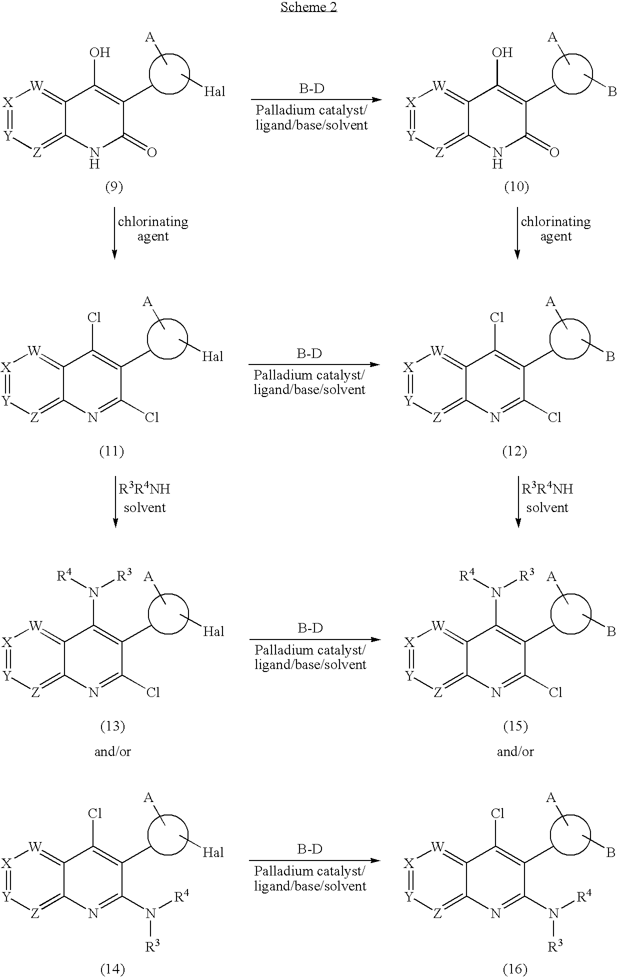

Compounds of formula (17) can be prepared as shown in Scheme 3 from compounds of formula (6) by reaction with a source of fluoride ion, such as potassium fluoride, in a suitable solvent such as sulpholane, at a temperature between 50° C. and 200° C., but preferably at 80-150° C. Compounds of formula (18) and/or compounds of formula (19) can be prepared from difluoro compounds of formula (17) by reaction with an amine of formula R³R⁴NH in a suitable solvent such as DMF or CH₂Cl₂, at a temperature of 0° C.-100° C., but preferably at room temperature.

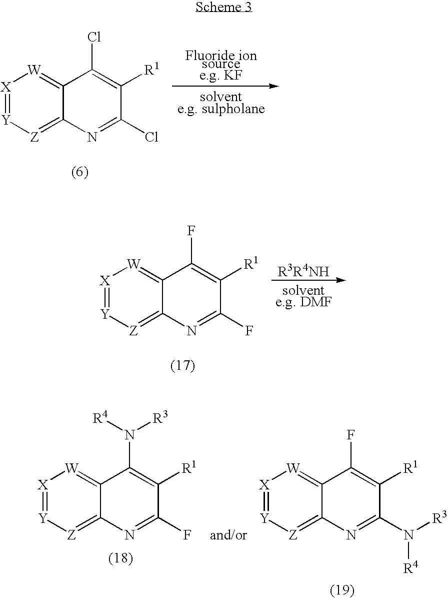

In Scheme 4 compounds of general formula (20), where the two R³R⁴N groups are identical, can be made from compounds of general formula (17) by reaction with a large excess of amine R³R⁴NH in a suitable solvent such as DMF, at a temperature between 0° C. and 150° C., but preferably between room temperature and 100° C.

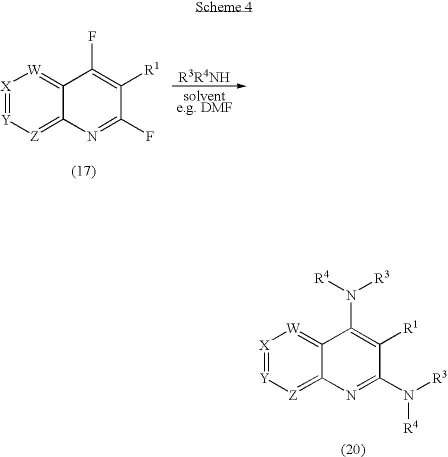

The intermediate chemicals having the general formulae (4), (5), (6), (9), (10), (11), (12), (13) (14) and (17):

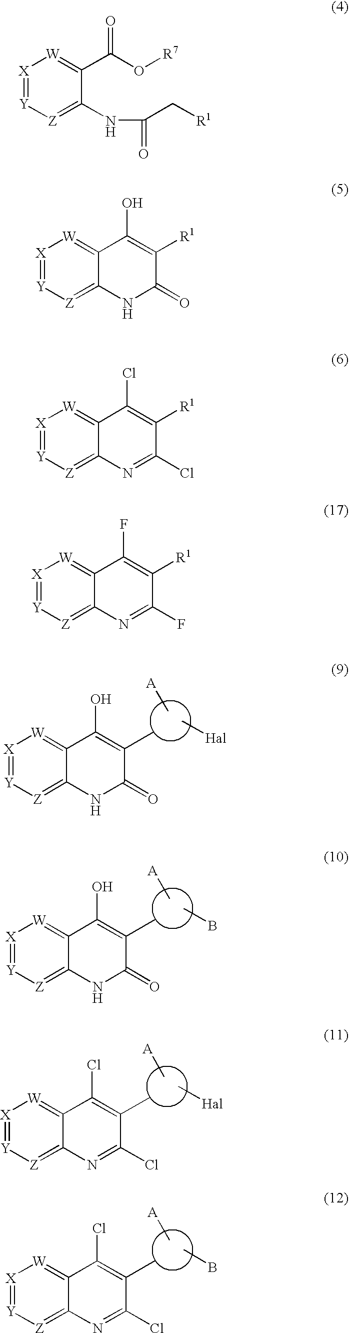

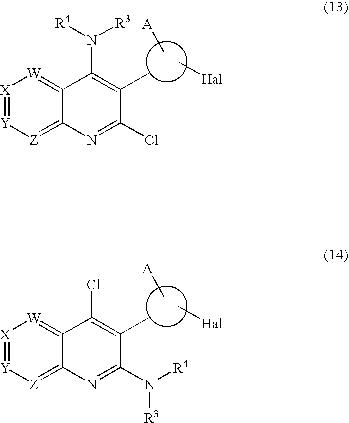

wherein W, X, Y, Z, R¹, R³, R⁴, R⁷, Hal, A and B are as defined above, are believed to be novel compounds and form a further part of this invention.

It should be noted that the intermediate of general formula (5) may exist in the tautomeric forms (a), (b) and (c) as well as in the form shown in formula (5):

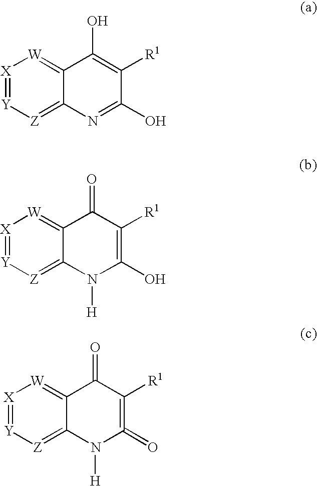

The invention as defined by the general formula (5) embraces all such tautomers.

Of particular interest are the intermediates listed in Tables below. In Table 28, the compounds have the general formula (4) where R¹ is R²⁰, R⁷ is methyl, W, X, Y, Z and B have the values shown in table 28.

TABLE 28

| Cmpd | | | | | |
|---|---|---|---|---|---|
| No. | B | W | X | Y | Z |
| 1 | phenyl | N | CH | CH | N |
| 2 | 4-fluorophenyl | N | CH | CH | N |
| 3 | 4-chlorophenyl | N | CH | CH | N |
| 4 | 5-fluoro-2-pyridyl | N | CH | CH | N |
| 5 | 6-fluoro-3-pyridyl | N | CH | CH | N |
| 6 | 2-phenylethenyl | N | CH | CH | N |
| 7 | 2-(4-fluorophenyl)ethenyl | N | CH | CH | N |
| 8 | (4-methylphenyl)ethynyl | N | CH | CH | N |
| 9 | (4-fluorophenyl)ethynyl | N | CH | CH | N |
| 10 | 4-fluorophenoxy | N | CH | CH | N |
| 11 | phenylthio | N | CH | CH | N |
| 12 | phenylsulphinyl | N | CH | CH | N |
| 13 | phenylsulphonyl | N | CH | CH | N |
| 14 | phenyl | N | CH | N | N |
| 15 | 4-fluorophenyl | N | CH | N | N |
| 16 | 4-chlorophenyl | N | CH | N | N |
| 17 | 5-fluoro-2-pyridyl | N | CH | N | N |
| 18 | 6-fluoro-3-pyridyl | N | CH | N | N |
| 19 | 2-phenylethenyl | N | CH | N | N |
| 20 | 2-(4-fluorophenyl)ethenyl | N | CH | N | N |
| 21 | (4-methylphenyl)ethynyl | N | CH | N | N |
| 22 | (4-fluorophenyl)ethynyl | N | CH | N | N |
| 23 | 4-fluorophenoxy | N | CH | N | N |
| 24 | phenylthio | N | CH | N | N |
| 25 | phenylsulphinyl | N | CH | N | N |
| 26 | Phenylsulphonyl | N | CH | N | N |

TABLE 29
Table 29 consists of 26 compounds of the general formula (5), where R¹
is R²⁰, W, X, Y, Z and B have the values given in Table 28 and R²⁰ is
4-substituted 2,6-difluorophenyl.

TABLE 30
Table 30 consists of 26 compounds of the general formula (5), where R¹
is R²⁰, W, X, Y, Z and B have the values given in Table 28 and R²⁰ is
4-substituted 2-chloro-6-fluorophenyl.

TABLE 31
Table 31 consists of 26 compounds of the general formula (5), where R¹
is R²⁰, W, X, Y, Z and B have the values given in Table 28 and R²⁰ is
4-substituted 2-chlorophenyl.

TABLE 32
Table 32 consists of 26 compounds of the general formula (5), where R¹
is R²⁰, W, X, Y, Z and B have the values given in Table 28 and R²⁰ is
5-substituted 3-fluoropyrid-2-yl.

TABLE 33
Table 33 consists of 26 compounds of the general formula (5), where R¹
is R²⁰, W, X, Y, Z and B have the values given in Table 28 and R²⁰ is
5-substituted 3-chloropyrid-2-yl.

TABLE 34
Table 34 consists of 26 compounds of the general formula (6), where R¹
is R²⁰, W, X, Y, Z and B have the values given in Table 28 and R²⁰ is
4-substituted 2,6-difluorophenyl.

TABLE 35
Table 35 consists of 26 compounds of the general formula (6), where R¹
is R²⁰, W, X, Y, Z and B have the values given in Table 28 and R²⁰ is
4-substituted 2-chloro-6-fluorophenyl.

TABLE 36
Table 36 consists of 26 compounds of the general formula (6), where R¹
is R²⁰, W, X, Y, Z and B have the values given in Table 28 and R²⁰ is
4-substituted 2-chlorophenyl.

TABLE 37
Table 37 consists of 26 compounds of the general formula (6), where R¹
is R²⁰, W, X, Y, Z and B have the values given in Table 28 and R²⁰ is
5-substituted 3-fluoropyrid-2-yl.

TABLE 38
Table 38 consists of 26 compounds of the general formula (6), where R¹
is R²⁰, W, X, Y, Z and B have the values given in Table 28 and R²⁰ is
5-substituted 3-chloropyrid-2-yl.

TABLE 39
Table 39 consists of 26 compounds of the general formula (17), where R¹
is R²⁰, W, X, Y, Z and B have the values given in Table 28 and R²⁰ is
4-substituted 2,6-difluorophenyl.

TABLE 40
Table 40 consists of 26 compounds of the general formula (17), where R¹
is R²⁰, W, X, Y, Z and B have the values given in Table 28 and R²⁰ is
4-substituted 2-chloro-6-fluorophenyl.

TABLE 41
Table 41 consists of 26 compounds of the general formula (17), where R¹
is R²⁰, W, X, Y, Z and B have the values given in Table 28 and R²⁰ is
4-substituted 2-chlorophenyl.

TABLE 42
Table 42 consists of 26 compounds of the general formula (17), where R¹
is R²⁰, W, X, Y, Z and B have the values given in Table 28 and R²⁰ is
5-substituted 3-fluoropyrid-2-yl.

TABLE 43
Table 43 consists of 26 compounds of the general formula (17), where R¹
is R²⁰, W, X, Y, Z and B have the values given in Table 28 and R²⁰ is
5-substituted 3-chloropyrid-2-yl.

TABLE 44
Table 44 consists of 26 compounds of the general formula (4), where R¹
is R²⁰, W, X, Y, Z and B have the values given in Table 28, R⁷ is
methyl and R²⁰ is 4-substituted 2,6-difluorophenyl.

TABLE 45
Table 45 consists of 26 compounds of the general formula (4), where R¹
is R²⁰, W, X, Y, Z and B have the values given in Table 28, R⁷ is
methyl and R²⁰ is 4-substituted 2-chloro-6-fluorophenyl.

TABLE 46
Table 46 consists of 26 compounds of the general formula (4), where R¹
is R²⁰, W, X, Y, Z and B have the values given in Table 28, R⁷ is
methyl and R²⁰ is 4-substituted 2-chlorophenyl.

TABLE 47
Table 47 consists of 26 compounds of the general formula (4), where R¹
is R²⁰, W, X, Y, Z and B have the values given in Table 28, R⁷ is
methyl and R²⁰ is 5-substituted 3-fluoropyrid-2-yl.

TABLE 48
Table 48 consists of 26 compounds of the general formula (4), where R¹
is R²⁰, W, X, Y, Z and B have the values given in Table 28, R⁷ is
methyl and R²⁰ is 5-substituted 3-chloropyrid-2-yl.

TABLE 49
Table 49 consists of 26 compounds of the general formula (4), where R¹ is
R²⁰, W, X, Y, Z and B have the values given in Table 28, R⁷ is ethyl and
R²⁰ is 4-substituted 2,6-difluorophenyl.

TABLE 50
Table 50 consists of 26 compounds of the general formula (4), where R¹ is
R²⁰, W, X, Y, Z and B have the values given in Table 28, R⁷ is ethyl and
R²⁰ is 4-substituted 2-chloro-6-fluorophenyl.

TABLE 51
Table 51 consists of 26 compounds of the general formula (4), where R¹ is
R²⁰, W, X, Y, Z and B have the values given in Table 28, R⁷ is ethyl and
R²⁰ is 4-substituted 2-chlorophenyl.

TABLE 52
Table 52 consists of 26 compounds of the general formula (4), where R¹ is
R²⁰, W, X, Y, Z and B have the values given in Table 28, R⁷ is ethyl and
R²⁰ is 5-substituted 3-fluoropvrid-2-yl.

TABLE 53
Table 53 consists of 26 compounds of the general formula (4), where R¹ is
R²⁰, W, X, Y, Z and B have the values given in Table 28, R⁷ is ethyl and
R²⁰ is 5-substituted 3-chloropyrid-2-yl.

The compounds of formula (1) are active fungicides and may be used to control one or more of the following pathogens: Pyricularia oryzae (Magnaporthe grisea) on rice and wheat and other Pyricularia spp. on other hosts; Puccinia triticiia (or recondita), Puccinia striiformis and other rusts on wheat, Puccinia hordei, Puccinia striiformis and other rusts on barley, and rusts on other hosts (for example turf, rye, coffee, pears, apples, peanuts, sugar beet, vegetables and ornamental plants); Erysiphe cichoracearum on cucurbits (for example melon); Blumeria (or Erysiphe) graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveillula taurica on tomatoes, aubergine and green pepper, Podosphaera leucotricha on apples and Uncinula necator on vines; Cochliobolus spp., Helminthosporium spp., Dreclslera spp. (Pyreiwphora spp.), Rhynchosporium spp., Mycosphaerella graminicola (Septoria tritici) and Phaeosphaeria nodorum (Stagotiospora nordorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Botrytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Gloinerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythiun ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucmeris on rice and turf and other Rhizoctonia spp. on various hosts such as wheat and barley, peanuts, vegetables, cotton and turf; Sclerotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; Laetisaria fuciformis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticilliun spp. on a range of hosts including hops, potatoes and tomatoes; Pyrenopeziza spp. on oil-seed rape and other hosts; Oncobasidium theobromae on cocoa causing vascular streak dieback; Fusarium spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicilliun italicum and Trichoderma viride on oranges, Colletotrichum musae and Gloeosporium musarum on bananas and Botrytis cinerea on grapes); other pathogens on vines, notably Eutypa lata, Guignardia bidwellii, Phellinus igniarus, Phoniopsis viticola, Pseudopeziza tracheiphila and Stereuin hirsutuin; other pathogens on trees (for example Lophlodennium seditiosum) or lumber, notably Cephaloascus fragrans, Ceratocystis spp., Ophiostoma piceae, Penicilliuin spp., Trichodenna pseudokoningii, Triczoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania).

A compound of formula (1) may move acropetally, basipetally or locally in plant tissue to be active against one or more fungi. Moreover, a compound of formula (1) may be volatile enough to be active in the vapour phase against one or more fungi on the plant.

The invention therefore provides a method of combating or controlling phytopathogenic fungi which comprises applying a fungicidally effective amount of a compound of formula (1), or a composition containing a compound of formula (1), to a plant, to a seed of a plant, to the locus of the plant or seed or to soil or any other plant growth medium, e.g. nutrient solution.

The term “plant” as used herein includes seedlings, bushes and trees. Furthermore, the fungicidal method of the invention includes protectant, curative, systemic, eradicant and antisporulant treatments.

The compounds of formula (1) are preferably used for agricultural, horticultural and turfgrass purposes in the form of a composition.

In order to apply a compound of formula (1) to a plant, to a seed of a plant, to the locus of the plant or seed or to soil or any other growth medium, a compound of formula (1) is usually formulated into a composition which includes, in addition to the compound of formula (1), a suitable inert diluent or carrier and, optionally, a surface active agent (SFA). SFAs are chemicals that are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (1). The composition is generally used for the control of fungi such that a compound of formula (1) is applied at a rate of from 0.1 g to 10 kg per hectare, preferably from 1 g to 6 kg per hectare, more preferably from 1 g to 1 kg per hectare.

When used in a seed dressing, a compound of formula (1) is used at a rate of 0.0001 to 10 g (for example 0.001 g or 0.05 g), preferably 0.005 g to 10 g, more preferably 0.005 g to 4 g, per kilogram of seed.

In another aspect the present invention provides a fungicidal composition comprising a fungicidally effective amount of a compound of formula (1) and a suitable carrier or diluent therefor.

In a still further aspect the invention provides a method of combating and controlling fungi at a locus, which comprises treating the fungi, or the locus of the fungi with a fungicidally effective amount of a composition comprising a compound of formula (1).

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (1).

Dustable powders (DP) may be prepared by mixing a compound of formula (1) with one or more solid diluents (for example natural clays, kaolin, pyrophyllite, bentonite, alumina, montmorillonite, kieselguhr, chalk, diatomaceous earths, calcium phosphates, calcium and magnesium-carbonates, sulphur, lime, flours, talc and other organic and inorganic solid carriers) and mechanically grinding the mixture to a fine powder.

Soluble powders (SP) may be prepared by mixing a compound of formula (1) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (1) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (1) and one or more powdered solid diluents or carriers, or from pre-formed blank granules by absorbing a compound of formula (1) (or a solution thereof, in a suitable agent) in a porous granular material (such as pumice, attapulgite clays, fuller's earth, kieselguhr, diatomaceous earths or ground corn cobs) or by adsorbing a compound of formula (1) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (1) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (1) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclo-hexanone), alcohols (such as benzyl alcohol, furfuryl alcohol or butanol), N-alkylpyr-rolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (1) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents that have a low solubility in water.

Microemulsions (ME) may be prepared by mixing water with a blend of one or more solvents with one or more SFAs, to produce spontaneously a thermodynamically stable isotropic liquid formulation. A compound of formula (1) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (1). SCs may be prepared by ball or bead milling the solid compound of formula (1) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (1) may be dry milled and added to water, containing agents hereinbefore described, to produce the desired end product.

Aerosol formulations comprise a compound of formula (1) and a suitable propellant (for example n-butane). A compound of formula (1) may also be dissolved or dispersed in a suitable medium (for example water or a water miscible liquid, such as n-propanol) to provide compositions for use in non-pressurised, hand-actuated spray pumps.

A compound of formula (1) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (1) and, optionally, a carrier or diluent therefor. The polymeric shell may be produced by either an interfacial polycondensation reaction or by a coacervation procedure. The compositions may provide for controlled release of the compound of formula (1) and they may be used for seed treatment. A compound of formula (1) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (1)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (1)).

A compound of formula (1) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecyl-benzenesulphonate, butylnaphthylene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SPAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (1) may be applied by any of the known means of applying fungicidal compounds. For example, it may be applied, formulated or unformulated, to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (1) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (1) (for example 0.0001 to 10%, by weight) depending upon the purpose for which they are to be used.

A compound of formula (1) may be used in mixtures with fertilizers (for example nitrogen-, potassium- or phosphorus-containing fertilizers). Suitable formulation types include granules of fertiliser. The mixtures suitably contain up to 25% by weight of the compound of formula (1).

The invention therefore also provides a fertiliser composition comprising a fertiliser and a compound of formula (1).

The compositions of this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than the compound of formula (1) alone. Further the other fungicide may have a synergistic effect on the fungicidal activity of the compound of formula (1).

The compound of formula (1) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of formula (1); or help to overcome or prevent the development of resistance to individual components. The particular additional active ingredient will depend upon the intended utility of the composition.

Examples of fungicidal compounds which may be included in the composition of the invention are AC 382042 (N-(1-cyano-1,2-dimethylpropyl)-2-(2,4-dichlorophenoxy) pro-pionamide), acibenzolar-5-methyl, alanycarb, aldimorph, anilazine, azaconazole, azafenidin, azoxystrobin, benalaxyl, benomyl, benthiavalicarb, biloxazol, bitertanol, blasticidin S, boscalid (new name for nicobifen), bromuconazole, bupirimate, captafol, captan, carbendazim, carbendazim chlorhydrate, carboxin, carpropamid, carvone, CGA 41396, CGA 41397, chinomethionate, chlorbenzthiazone, chlorothalonil, chlorozolinate, clozylacon, copper containing compounds such as copper oxychloride, copper oxyquinolate, copper sulphate, copper tallate, and Bordeaux mixture, cyamidazosulfamid, cyazofamid (IKF-916), cyflufenamid, cymoxanil, cyproconazole, cyprodinil, debacarb, di-2-pyridyl disulphide 1,1′-dioxide, dichlorfluanid, diclocymet, diclomezine, dicloran, diethofencarb, difenoconazole, difenzoquat, diflumetorim, O,O-di-iso-propyl-5-benzyl thiophosphate, dimefluazole, dimetconazole, dimethirimol, dimethomorph, dimoxystrobin, diniconazole, dinocap, dithianon, dodecyl dimethyl ammonium chloride, dodemorph, dodine, doguadine, edifenphos, epoxiconazole, ethaboxam, ethirimol, ethyl (Z)-N-benzyl-N([methyl(methyl-thioethylideneaminooxycarbonyl)amino]thio)-β-alaninate, etridiazole, famoxadone, fenamidone, fenarimol, fenbuconazole, fenfuram, fenhexamid, fenoxanil (AC 382042), fenpiclonil, fenpropidin, fenpropimorph, fentin acetate, fentin hydroxide, ferbam, ferimzone, fluazinam, fludioxonil, flumetover, flumorph, fluoroimide, fluoxastrobin, fluquinconazole, flusilazole, flusulfamide, flutolanil, flutriafol, folpet, fosetyl-aluminium, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb, isopropanyl butyl carbamate, isoprothiolane, kasugamycin, kresoxim-methyl, LY186054, LY211795, LY 248908, mancozeb, maneb, mefenoxam, mepanipyrim, mepronil, metalaxyl, metalaxyl M, metconazole, metiram, metiram-zinc, metominostrobin, metrafenone, MON65500 (N-allyl-4,5-dimethyl-2-trimethylsilylthiophene-3-carboxamide), myclobutanil, NTN0301, neoasozin, nickel dimethyldithiocarbamate, nitrothale-isopropyl, nuarimol, ofurace, organomercury compounds, orysastrobin, oxadixyl, oxasulfuron, oxolinic acid, oxpoconazole, oxycarboxin, pefurazoate, penconazole, pencycuron, phenazin oxide, phosphorus acids, phthalide, picoxystrobin, polyoxin D, polyram, probenazole, prochloraz, procymidone, propamocarb, propamocarb hydrochloride, propiconazole, propineb, propionic acid, proquinazid, prothioconazole, pyraclostrobin, pyrazophos, pyrifenox, pyrimethanil, pyroquilon, pyroxyfur, pyrrolnitrin, quaternary ammonium compounds, quinomethionate, quinoxyfen, quintozene, silthiofam (MON 65500), S-imazalil, simeconazole, sipconazole, sodium pentachlorophenate, spiroxamine, streptomycin, sulphur, tebuconazole, tecloftalam, tecnazene, tetraconazole, thiabendazole, thifluzamide, 2-(thiocyanomethylthio)-benzothiazole, thiophanate-methyl, thiram, tiadinil, timibenconazole, tolclofos-methyl, tolylfluanid, triadimefon, triadimenol, triazbutil, triazoxide, tricyclazole, tridemorph, trifloxystrobin, triflumizole, triforine, triticonazole, validamycin A, vapam, vinclozolin, XRD-563, zineb, ziram, zoxamide and compounds of the formulae:

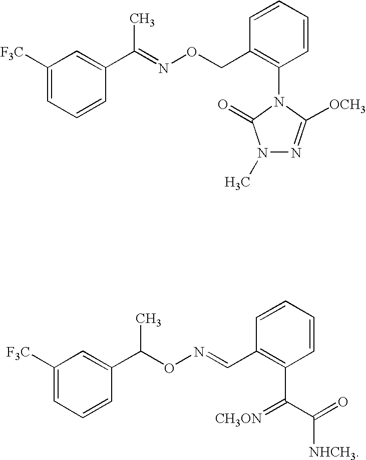

The compounds of formula (1) may be mixed with soil, peat or other rooting media for the protection of plants against seed-borne, soil-borne or foliar fungal diseases.

Some mixtures may comprise active ingredients, which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples in which the following abbreviations are used:

ml = millilitres
g = grammes
ppm = parts per million
s = singlet
d = doublet
t = triplet
q = quartet
m = multiplet
b = broad
f = fine
THF = tetrahydrofuran
DCM = dichloromethane
DMF = N,N-dimethylformamide
DMSO = dimethylsulphoxide
DMAP = 4-dimethylaminopyridine
NMR = nuclear magnetic resonance
HPLC = high performance liquid chromatography

### EXAMPLES

### Example 1

This example illustrates the preparation of sec-butyl-[6-chloro-7-[(4-fluorophenyl)-2,6-difluorophenyl]-pyrido[2,3-b]pyrazin-8-yl]-amine of the formula below, Compound 3.002.

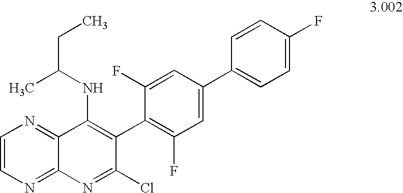

Step 1: The Preparation of 2,6-difluoro-4-bromobenzyl methanesulphonate

2,6-difluoro-4-bromobenzyl alcohol (9.50 g) and triethylamine (5.0 g) were dissolved in THF cooled to 10° C. with stirring. Methanesulphonyl chloride (4.8 g) was added in a solution of THF (10 ml) over 10 minutes, and a white solid precipitated from the solution. The reaction was then warmed to room temperature for one hour and then the solid was collected and washed with diethyl ether. The filtrate was evaporated to give 2,6-difluoro-4-bromobenzyl methanesulphonate (13.0 g) as a golden oil which slowly crystallised.

¹H NMR (CDCl₃) 8 ppm: 3.05 (s, 3H), 5.3 (s, 2H), 7.15 (t, 2H).

Step 2: The Preparation of 2,4-difluoro-4-bromobenzyl Cyanide

Potassium cyanide (2.8 g) was dissolved in water and was added to a stirred solution of the product of Step 1 (13.0 g) in ethanol (100 ml). The reaction was refluxed for 2-hours, and was then cooled and the solvent evaporated to give a sludge. Water was added and the mixture was extracted with DCM and dried over magnesium sulphate. The solution was evaporated and to give a sludge which was triturated with a small amount of diethyl ether to give 2,4-difluoro-4-bromobenzyl cyanide as a light brown solid (5.2 g).

¹H NMR (CDCl₃) 8 ppm: 3.7 (s, 2H), 7.18 (t, 2H).

Step 3: The Preparation of 2,6-difluoro-4-bromophenyl Acetic Acid

The product from Step 2 (4.2 g) was dissolved in a mixture of water (25 ml) and concentrated sulphuric acid (25 ml), and the reaction was refluxed for 3 hours. The reaction was then cooled and the solid collected was washed with water and dried to give 2,6-difluoro-4-bromophenyl acetic acid as a light brown crystalline solid (3.8 g).

¹H NMR (CDCl₃) δ ppm: 3.75 (s, 2H), 7.1 (t, 2H).

Step 4: The Preparation of 2,6-difluoro-4-bromophenyl acetyl chloride

The product from Step 3 (3.6 g) was added portionwise to thionyl chloride (10 ml), pre-heated to 60° C., with two drops of DMF. Reaction was immediate and after the addition the reaction was refluxed for a further 1 hour, and was then cooled and evaporated to give the acid chloride as a brown liquid (3.6 g), which was used in the next reaction without further purification.

Step 5: The Preparation of Methyl 3-[2-(4-bromo-2,6-difluoro-phenyl)-acetyl amino]-pyrazine-2-carboxylate:

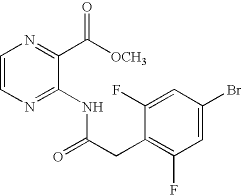

A solution of the crude acid chloride from Step 4 (3.6 g) in DCM (10 ml) was added dropwise to a stirred solution of methyl 2-aminopyrazine carboxylate (2.2 g) and pyridine (5 ml) stirred at 10° C. in DCM. The reaction was stirred for 15 hours at room temperature, and the solvent was evaporated and water was added, followed by extraction with ethyl acetate. The organic fraction was washed with water and aqueous sodium carbonate, followed by dilute hydrochloric acid. The ethyl acetate was dried over magnesium sulphate and evaporated to give a dark sludge, which was triturated with diethyl ether, and methyl 3-[2-(4-bromo-2,6-difluoro-phenyl)-acetylamino]-pyrazine-2-carboxylate was isolated as a buff solid (2.9 g).

¹H NMR (CDCl₃) δ ppm: 4.0 (s, 2H), 4.05 (s, 3H), 7.15 (dd, 2H), 8.4 (d, 1H), 8.6 (d, 1H).

Step 6: The Preparation of 7-(4-bromo-2,6-difluoro-phenyl)-8-hydroxy-5H-pyrido[2,3-]pyrazin-6-one:

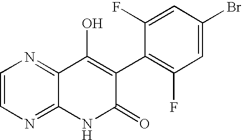

The product from Step 5 (2.9 g) and potassium carbonate (2.1 g) in dry DMF (20 ml) were heated to 100° C. (oil bath) for 4 hours, giving a yellow suspension. The solvent was evaporated to dryness and the dark sludge was triturated with diethyl ether, and the pale green solid collected. This solid was dissolved in water and acidified with 4M hydrochloric acid, and the precipitated solid was collected and dried to give 7-(4-bromo-2,6-difluoro-phenyl)-8-hydroxy-5H-pyrido[2,3-]pyrazin-6-one as a buff solid (1.6 g).

¹H NMR (CDCl₃) δ ppm: 7.3 (d, 2H), 8.55 (d, 1H), 8.65 (d, 1H).

Step 7: The Preparation of 7-(4-bromo-2,6-difluoro-phenyl)-6,8-dichloro-pyrido[2,3-b]pyrazine

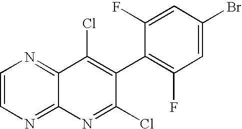

The product from Step 6 (0.353 g) and phosphorus oxychloride (3 ml) were mixed together at room temperature and then refluxed with stirring for 6 hours. The reaction was cooled and evaporated to dryness and water and DCM were added.

The DCM extract was washed with aqueous sodium carbonate, dried with magnesium sulphate, and evaporated to give an oil which was purified by flash column chromatography on silica gel eluting with diethyl ether to give 7-(4-bromo-2,6-difluoro-phenyl)-6,8-dichloro-pyrido[2,3-b]pyrazine as a reddish solid (0.205 g), which was used without further purification.

Step 8: The Preparation of [7-(4-Bromo-2,6-difluoro-phenyl)-6-chloro-pyrido[2,3-b]pyrazin-8-yl]-sec-butyl-amine

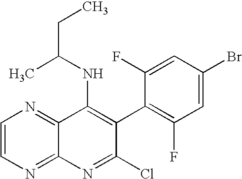

The product from Step 7 (0.205 g) and s-butylamine (1.0 ml) were mixed together in a sealed tube and were stirred at room temperature for 4 days. The reaction mixture was evaporated to give a sludge, which was then purified by flash column chromatography on silica gel eluting with diethyl ether:hexane 1:2, and then diethyl ether:hexane 4:1, to give [7-(4-Bromo-2,6-difluoro-phenyl)-6-chloro-pyrido[2,3-b]pyrazin-8-yl]-sec-butyl-amine as a yellow solid (0.095 g).

¹H NMR (CDCl₃) δ ppm: 0.8 (t, 3H), 1.1 (d, 3H), 1.45 (m, 2H), 3.15 (m, 2H), 6.95 (bd, 1H), 7.3 (d, 2H), 8.7 (d, 1H), 9.0 (d, 1H).

Step 9: The preparation of sec-butyl-[6-chloro-7-[(4-fluorophenyl)-2,6-difluorophenyl]-pyrido[2,3-b]pyrazin-8-yl)-amine, Compound 3.002

The product from Step 8 (0.027 g), 4-fluorophenyl boronic acid (0.012 g), potassium carbonate (0.020 g) and tetrakis(triphenylphosphine)palladium (0.001 g) were mixed and brought to reflux for 6 hours in toluene (2.0 ml). The reaction was cooled and evaporated and the crude product was dissolved in diethyl ether and then purified by flash column chromatography on silica gel eluting with diethyl ether, to give the title product as a white gum (0.010 g).

¹H NMR (CDCl₃) δ ppm: 0.7 (t, 3H), 1.1 (d, 3H), 1.45 (m, 2H), 3.2 (m, 1H), 6.95 (bd, 1H), 7.15 (d, 2H), 7.2 (d, 2H), 7.65 (m, 2H), 8.7 (d, 1H), 9.0 (d, 1H).

### Example 2

This example illustrates the preparation of sec-butyl-[6-chloro-7-[phenyl-2,6-difluorophenyl]-pyrido[2,3-b]pyrazin-8-yl]-amine, Compound 2.002

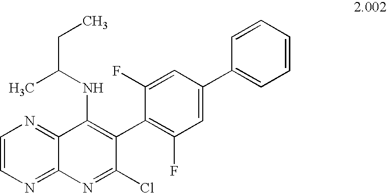

The compound was prepared analogous to Step 9 from Example 1 from the product of Step 8 of Example 1, but the coupling reaction was carried out with phenyl boronic acid instead of 4-fluorophenyl boronic acid.

¹H NMR (CDCl₃) δ ppm: 0.75 (t, 3H), 1.1 (d, 3H), 1.45 (m, 2H), 3.2 (m, 1H), 6.95 (bd, 1H), 7.3 (d, 2H), 7.45-7.5 (m, 3H), 7.65 (d, 2H), 8.65 (fd, 1H), 9.0 (fd, 1H).

### Example 3

This example illustrates the preparation of sec-butyl-[6-chloro-7-(2,6-difluoro-(4-methylphenylethynyl)-phenyl)-pyrido[2,3-b]pyrazin-8-yl]-amine of the formula below, Compound 9.002.

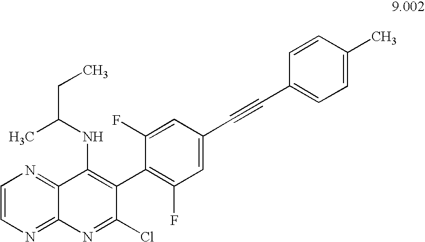

[7-(4-Bromo-2,6-difluoro-phenyl)-6-chloro-pyrido[2,3-b]pyrazin-8-yl]-sec-butyl-amine (0.030 g), 4-methylphenyl acetylene (0.016 g), cuprous iodide (0.001 g), dichlorobis (triphenylphosphine)palladium (0.003 g) and triethylamine (5 ml) were refluxed for 7 hours. The reaction was cooled and evaporated to give a sludge, which was taken up in diethyl ether and purified by flash column chromatography on silica gel eluting with diethyl ether, to give the title compound as a gum (0.008 g).

¹H NMR (CDCl₃) δ ppm: 0.75 (t, 3H), 1.05 (d, 3H), 1.45 (m, 1H), 2.4 (s, 3H), 3.15 (m, 1H), 6.95 (bd, 1H), 7.2 (d, 4H), 7.45 (d, 2H), 8.15 (bs, 1H), 9.0 (bs, 1H).

### Example 4

### This Example Illustrates the Fungicidal Properties of the Compounds of the General Formula (1)

Septoria tritici (leaf blotch): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours.

Pyricularia orzyae (rice blast): Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hours.

The following compounds gave greater than 60% control of disease:

Septoria tritici: 2.002
Pyricularia orzyae: 2.002

1. The compound of the general formula (1): 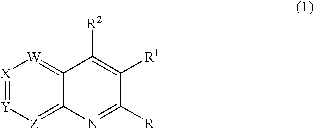
wherein
W, X, Y and Z can be N or CR⁸, with at least one and no more than three of W, X, Y and Z being N, but excluding compounds where W, X, Y═N and Z=CR⁸, and X, Y, Z=N and Z=CR⁸;
R⁸ is H, halo, C₁₋₄ alkyl, C₁₋₄ alkoxy or halo(C₁₋₄)alkyl, CN, C₁₋₄alkylthio, C₁₋₄alkylsulphinyl, C₁₋₄alkylsulphonyl, aryl, heteroaryl, halo(C₁₋₆)alkoxy, halo(C₁₋₄)alkylthio, C₂₋₄alkenyl, C₂₄₋₆alkynyl, C₂₋₆cycloalkyl, or NR³R⁴;
R is H, C₁₋₄ alkyl, halo(C₁₋₄)alkyl, cyano, halogen or NR³R⁴;
R² is halo or NR³R⁴;
R¹ is an aryl or heteroaryl ring R²⁰, of the general formula 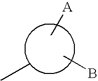
where A can be one to four optional substituents independently selected from halo, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, C₁₋₄alkoxy(C₁₋₆)alkyl, C₃₋₆ cycloalkyl, C₃₋₆ cycloalkyl(C₁₋₄)alkyl, and B is at least one or more substituents independently selected from aryl, heteroaryl, aryloxy (except that phenoxy must be substituted), heteroaryloxy, aryl(C₁₋₄)alkoxy (except that benzyloxy must be substituted), heteroaryl(C₁₋₄)alkoxy, arylthio, arylsulphinyl, arylsulphonyl, heteroarylthio, heteroarylsulphinyl, heteroarylsulphonyl, aryl(C₂₋₄)alkenyl, aryl(C₂₋₄)alkynyl, heteroaryl(C₂₋₄)alkenyl, heteroaryl(C₂₋₄)alkynyl, aryl(C₁₋₄)alkyl, heteroaryl(C₁₋₄)alkyl, with any of the forgoing aryl or heteroaryl substituents being optionally substituted with halo, C₁₋₆ alkyl, C₂₋₆ alkenyl, C₂₋₆ alkynyl, C₁₋₆alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)-alkylthio, C₁₋₄ alkoxy(C₁₋₆)alkyl, C₃₋₆ cycloalkyl, cyano or nitro;
R³ and R⁴ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl, heteroaryl(C₁₋₈)alkyl, NR⁵R⁶ or
R³ and R⁴ together form a C₃₋₇ alkylene or C₃₋₇ alkenylene chain optionally substituted with one or more C₁₋₄ alkyl or C₁₋₄ alkoxy groups, or,
together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl ring, and R⁵ and R⁶ are independently H, C₁₋₈ alkyl, C₂₋₈ alkenyl, C₂₋₈ alkynyl, aryl, aryl(C₁₋₈)alkyl, C₃₋₈ cycloalkyl, C₃₋₈ cycloalkyl(C₁₋₆)alkyl, heteroaryl or heteroaryl(C₁₋₈)alkyl; any of the foregoing alkyl, alkenyl, alkynyl or cycloalkyl groups or moieties (other than for R⁸) being optionally substituted with halogen, cyano, C₁₋₆ alkoxy, C₁₋₆ alkylcarbonyl, C₁₋₆ alkoxycarbonyl, C₁₋₆ haloalkoxy, C₁₋₆ alkylthio, tri(C₁₋₄)alkylsilyl, C₁₋₆ alkylamino or C₁₋₆ dialkylamino, any of the foregoing morpholine, thiomorpholine, piperidine, piperazine and pyrrolidine rings being optionally substituted with C₁₋₄ alkyl (especially methyl), and any of the foregoing aryl or heteroaryl groups or moieties in R³, R⁴, R⁵, R⁶ or R⁸ being optionally substituted with one or more substituents selected from halo, hydroxy, mercapto, C₁₋₆ alkyl, C₂₋₆ alkenyl, C₂₋₆ alkynyl, C₁₋₆ alkoxy, C₂₋₆ alkenyloxy, C₂₋₆ alkynyloxy, halo(C₁₋₆)alkyl, halo(C₁₋₆)alkoxy, C₁₋₆ alkylthio, halo(C₁₋₆)alkylthio, hydroxy(C₁₋₆)alkyl, C₁₋₄ alkoxy(C₁₋₆)alkyl, C₃₋₆ cycloalkyl, C₃₋₆ cycloalkyl(C₁₋₄)alkyl, phenoxy, benzyloxy, benzoyloxy, cyano, isocyano, thiocyanato, isothiocyanato, nitro, —NR¹³R¹⁴, —NHCOR¹³, —NHCONR¹³R¹⁴, —CONR¹³R¹⁴, —SO₂R¹³, —OSO₂R¹³—COR¹³, —CR¹³═NR¹⁴ or —N═CR¹³R¹⁴ in which R¹³ and R¹⁴ are independently hydrogen, C₁₋₄ alkyl, halo(C₁₋₄)alkyl, C₁₋₄ alkoxy, halo(C₁₋₄)alkoxy, C₁₋₄ alkylthio, C₃₋₆ cycloalkyl, C₃₋₆ cycloalkyl(C₁₋₄)alkyl, phenyl or benzyl, the phenyl and benzyl groups being optionally substituted with halogen, C₁₋₄ alkyl or C₁₋₄ alkoxy.

2. A compound according to claim 1 wherein W and Z are N and X and Y are CH.

3. A compound according to claim 1 wherein R² is NR³R⁴.

4. A compound according to claim 3 wherein R is halo.

5. A compound according to claim 1 wherein
R³ is C₁₋₈ alkyl, halo(C₁₋₈)alkyl, haloC₁₋₄ alkoxy(C₁₋₈)alkyl, C₁₋₄ alkoxyhalo(C₁₋₈)alkyl C₁₋₄ alkoxycarbonyl(C₁₋₈)alkyl, C₁₋₄ alkoxycarbonylhalo(C₁₋₈)alkyl, phenyl(₁₋₄)alkyl, C₂₋₈ alkenyl, halo(C₂₋₈)alkenyl, C₂₋₈ alkynyl, C₃₋₈ cycloalkyl optionally substituted with chloro, fluoro or methyl, C₃₋₈ cycloalkyl(C₁₋₄)alkyl, phenylamino, piperidino or morpholino, the phenyl ring of phenylalkyl or phenylamino being optionally substituted with one, two or three substituents selected from halo, C₁₋₄ alkyl, halo(C₁₋₄)alkyl, C₁₋₄ alkoxy and halo(C₁₋₄)alkoxy; and
R⁴ is H, C₁₋₄ alkyl, halo(C₁₋₄)alkyl or amino, or
R³ and R⁴ together form a C₃₋₇ alkylene or alkenylene chain optionally substituted with methyl, or,
together with the nitrogen atom to which they are attached, R³ and R⁴ form a morpholine, thiomorpholine, thiomorpholine S-oxide or thiomorpholine S-dioxide ring or a piperazine or piperazine N—(C₁₋₄)alkyl (especially N-methyl) ring, in which the morpholine or piperazine rings are optionally substituted with methyl.

6. A process for preparing a compound of the general formula (1) according to claim 1 wherein one of R and R² is chloro or fluoro and the other is NR³R⁴ and W, X, Y, Z, R¹, R³ and R⁴ are as defined in claim 1, which comprises reacting an amine of the general formula NR³R⁴ with a compound of the general formula (6) or (17):

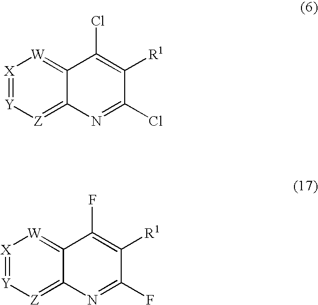

7. The intermediate chemicals having the general formulae (4), (5), (6), (9), (10), (11), (12), (13) (14) and (17): 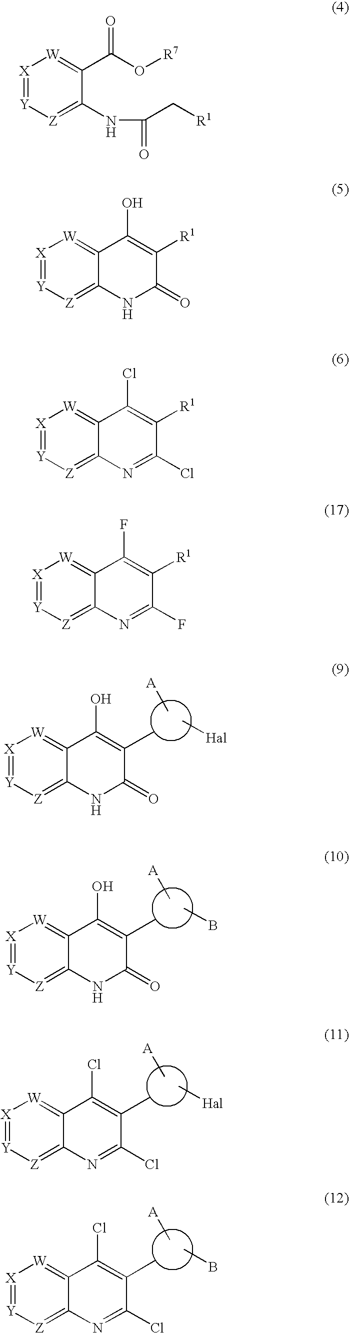 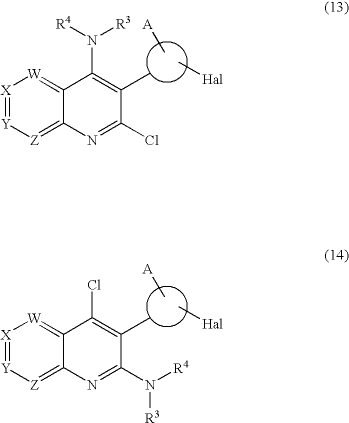
wherein W, X, Y, Z, R¹, R³, R⁴, Hal, A and B are as defined in claim 1 and R⁷, is C₁₋₄ alkyl.

8. A plant fungicidal composition comprising a fungicidally effective amount of a compound as defined in claim 1 and a suitable carrier or diluent therefor.

9. A method of combating or controlling phytopathogenic fungi which comprises applying to a plant, to a seed of a plant, to the locus of the plant or seed or to soil or to any other plant growth medium, a fungicidally effective amount of a compound according to claim 1 or a composition including said compound.
